SEQUENCE LISTING (6 sequences)

SEQ ID NO: 1 - DNA, 72 bp, Artificial Sequence
  Description of Artificial Sequence Synthetic oligonucleotide
aggcaccatcgtaggtttcttgccaggcaccatcgtaggtttcttgccaggcaccatcgtaggtttcttgcc

SEQ ID NO: 2 - DNA, 39 bp, Artificial Sequence
  Description of Artificial Sequence Synthetic oligonucleotide
cagaggcgctgcaagcctacgatggacacggtaacgact

SEQ ID NO: 3 - DNA, 50 bp, Artificial Sequence
  Description of Artificial Sequence Synthetic oligonucleotide
agcaacctgcctgttagcgcctctgttttttccatgacgttcctgacgtt

SEQ ID NO: 4 - DNA, 23 bp, Artificial Sequence
  Description of Artificial Sequence Synthetic oligonucleotide
ttaccgtgtggttgctagtcgtt

SEQ ID NO: 5 - DNA, 20 bp, Artificial Sequence
  Description of Artificial Sequence Synthetic oligonucleotide
tccatgacgttcctgacgtt

SEQ ID NO: 6 - DNA, 20 bp, Artificial Sequence
  Description of Artificial Sequence Synthetic oligonucleotide
tccatgacgttcctgacgtt

(19) United States
(12) Patent Application Publication
(10) Pub. No.: US 2015/0004193 A1
(43) Pub. Date: Jan. 1, 2015

(54) NOVEL DNA-ORIGAMI NANOVACCINES

(71) Applicant: ARIZONA BOARD OF REGENTS, A BODY CORPORATE OF THE STATE OF ARIZONA, ACTING FOR AND ON BEHALF OF ARIZ, Scottsdale, AZ (US)

(72) Inventors: Yung Chang, Tempe, AZ (US); Hao Yan, Chandler, AZ (US); Giovanna Ghirlanda, Scottsdale, AZ (US)

(21) Appl. No.: 14/376,814

(22) PCT Filed: Feb. 6, 2013
(86) PCT No.: PCT/US13/24945

§ 371 Date: Aug. 5, 2014

Related U.S. Application Data
(60) Provisional application No. 61/595,501, filed on Feb. 6, 2012.

(51) Int. Cl. A61K 47/48; A61K 39/21; A61K 31/7088

(52) U.S. Cl. CPC A61K 47/4823; A61K 47/48346; A61K 47/48723; A61K 31/7088; A61K 39/21
USPC 424/193.1; 536/24.5; 536/23.1; 514/44 A; 514/44 R; 530/395

(57) ABSTRACT

The present invention provides compositions comprising a DNA-nanostructure and at least one targeting moiety, wherein the at least one targeting moiety is linked to the DNA-nanostructure; and wherein the at least one targeting moiety is selected from the group consisting of antigens, aptamers, shRNAs and combinations thereof, and methods of use thereof.

### RELATED APPLICATION

This patent application claims the benefit of priority of U.S. application Ser. No. 61/595,501, filed Feb. 6, 2012, which application is incorporated by reference herein.

### STATEMENT REGARDING FEDERALLY SPONSORED RESEARCH

This invention was made with government support under grants CA141021 and DA 030045 awarded by the National Institutes of Health. The government has certain rights in the invention.

### BACKGROUND OF THE INVENTION

Substance abuse is known to contribute to the transmission of human immunodeficiency virus type 1 (HIV-1) among adolescents and young adults. While a high HIV prevalence among IV-drug users is caused by direct exposure to HIV-contaminated blood through needle sharing, many drug users, including those using non-injecting substances, may also acquire HIV through risky sexual behaviors influenced by illicit drugs. Despite some success of several HIV prevention programs, such as clean needle exchange and safe-sex education, and powerful anti-retroviral drugs in reducing HIV transmission, an HIV vaccine may ultimately be the best option for eradicating HIV/AIDS in high-risk drug user populations. Given the extremely high mutation rate of HIV genomes, only the prophylactic HIV vaccines that can induce immunity at the portal of entry would be considered valuable in controlling HIV infection. Despite three decades of extensive effort, such vaccines are still not yet within reach and current strategies for vaccine development suffer from either safety issues or ineffectiveness. The modest success of the recent Thai RV-144 clinical trial, which only offered 31% protection from HIV transmission among high risk groups, highlights this urgent need for new strategies in designing HIV vaccines.

Accordingly, new strategies and approaches for vaccine and therapeutic development are needed. In particular, new HIV and cancer vaccines and therapeutics are needed.

### SUMMARY OF THE INVENTION

The present invention provides a composition comprising a DNA-nanostructure and at least one targeting moiety, wherein the at least one targeting moiety is linked to the DNA-nanostructure; and wherein the at least one targeting moiety is selected from the group consisting of antigens, aptamers, shRNAs and combinations thereof. In certain embodiments, the DNA-nanostructure is selected from a biotin-oligo (i.e., oligonucleotide is biotinylated), a DNA-tetrahedron and a DNA-branch. In certain embodiments, the DNA-branch comprises four oligonucleotides. In certain embodiments, the composition further comprises at least one adjuvant, wherein the adjuvant is linked to the DNA nanostructure. In certain embodiments, the adjuvant is a CpG motif. In certain embodiments, the at least one adjuvant is an oligonucleotide containing at least one immunostimulatory CpG motif. In certain embodiments, the oligonucleotide is from about 8-30 bases in length. In certain embodiments, the antigen is selected from the group consisting of B-cell epitopes, T-cell epitopes, Tₕₑₗₚₑᵣ epitopes, epitopes derived from HIV gp120, gp41 epitopes, glycans, as well as other peptides, T-helper peptides, and streptavidin. In certain embodiments, the antigen binds to a neutralizing antibody or an inhibitory antibody. In certain embodiments, the antigen is a neutralizing epitope, such as a gp120 epitope, gp41 epitope or a CD4b epitope. In certain embodiments, the neutralizing epitope is a peptide that mimics the CD4 binding site (CD4b). In certain embodiments, the peptide binds to the neutralizing antibody b12. In certain embodiments, the neutralizing epitope is a glycan that binds to the neutralizing antibody 2G12. In certain embodiments, the at least one targeting moiety is an aptamer. In certain embodiments, the aptamer binds to an HIV epitope (such as a gp120 epitope), or a cell surface receptor expressed on an immune cell. In certain embodiments, the cell surface receptor is CD16 or cytotoxic T-lymphocyte antigen 4 (CTLA4). In certain embodiments, the at least one targeting moiety is shRNA, such as a Foxop3-shRNA.

In certain embodiments, the composition comprises at least two targeting moieties (e.g. 2, 3, 4, 5, 6, 7, 8, 9, 10, etc.). In certain embodiments, the targeting moieties are the same. In certain embodiments, the targeting moieties are different. In certain embodiments, one targeting moiety is a glycan that binds to the neutralizing antibody 2G12 and the other targeting moiety is a peptide that binds to the neutralizing antibody b12. In certain embodiments, the composition further comprises at least one T-helper peptide and at least one adjuvant, wherein the T-helper peptide and the adjuvant are linked to the DNA nanostructure. In certain embodiments, the adjuvant is an oligonucleotide containing at least one CpG motif. In certain embodiments, one targeting moiety is a first aptamer and the other targeting moiety is a second aptamer. In certain embodiments, the first aptamer binds an HIV infected cell and the second aptamer binds to an immune cell. In certain embodiments, the first aptamer binds to a gp120 epitope. In certain embodiments, the second aptamer binds to CD 16. In certain embodiments, one targeting moiety is an aptamer and the other targeting moiety is shRNA.

The present invention provides a composition as described above in combination with a physiologically-acceptable, non-toxic vehicle.

The present invention provides a method of inducing an immune response in a subject, comprising administering to the subject a therapeutically effective amount of the composition described above.

The present invention provides a method of inducing the production of high affinity neutralizing antibodies or inhibitory antibodies comprising administering the composition described above to a subject having a pathological condition.

The present invention provides a method of inducing a therapeutic immune response in a subject having or at risk of having a pathological condition, comprising administering to the subject a therapeutically effective amount of the composition of the composition described above.

The present invention provides a method for treating a subject with a pathological condition comprising administering a therapeutically effective amount of the composition as described above to the subject.

The present invention provides the use of a composition as described above for the manufacture of a medicament useful for the treatment of a pathological condition in a subject. In certain embodiments, the subject is a mammal, such as a human.

The present invention provides a composition as described above for use in the prophylactic or therapeutic treatment of a pathological condition. In certain embodiments, the pathological condition is human immunodeficiency virus (HIV).

The present invention provides a composition as described above for use in therapy.

### BRIEF DESCRIPTION OF THE FIGURES

FIG. 1. Illustration of the assembly of a model antigen (streptavidin) and an immunoadjuvant CpG oligonucleotide onto a DNA nanostructure. CpG oligodeoxynucleotides (CpG ODN) are short single stranded (ss) DNA that contain “C—P(phosphodiester or phosphorothioate)-G” structure. Three other ss DNA are synthesized to have partial complementary sequences, one of which contains biotin that allows subsequent binding to streptavidin. All four DNA strands are assembled by heating at 95° C. and then annealing at room temperature.

FIGS. 2A-2B. Schematic illustration of HIV-DNA-nanovaccines and their potential immunogenicity. A. Structure of HIV DNA-nanoparticle containing HIV epitopes, Tₕₑₗₚₑᵣ epitopes and CpG-DNA. B. Predicted anti-gp120 antibody responses induced by HIV DNA-nanoparticles. The gp120-DNA nanoparticles bound specifically to B cells or nonspecifically to dendritic cells (DC) are internalized by both B cells and DCs, and the peptides can be presented by MHC-II to T cells that are specific to the conjugated peptides. Illustration is not in scale.

FIGS. 3A-3C. Anti-streptavidin (STV) antibody responses in mice immunized with different forms of STV. The direct Biotin-CpG-STV serves a positive control. A. Immunization schedule. B. Conjugating CpG ODN and antigen to the J1 DNA nanostructure stimulated higher antibody response in vivo as compared to free CpG and antigen. C. Lack of anti-dsDNA antibody responses in mice immunized DNA-scaffolded STV. Relative OD was derived by calculation each sample against the negative control.

FIG. 4. Conjugated CpG showed higher cellular uptake in vivo.

FIG. 5. Different DNA nanostructures for in vitro and in vivo tests (Zhang, et al., Chem Commun, 46, 6792-6794 (2010).

FIG. 6. DNA nanostructures are stable in cell culture medium. “M” indicates 100 bp DNA ladder.

FIG. 7. DNA nanostructures enhance cellular uptake in the mouse macrophage-like cell line (RAW cells).

FIGS. 8A-8B. Evaluation of neutralizing epitopes. A. Experimental strategies for evaluation of neutralizing epitopes and their immunogenicity. B. ELISA for examining the structure of constructed epitopes.

FIG. 9. Illustration of the construction platform of HIV-DNA origami antibody vaccines.

FIG. 10. Illustration of targeted destruction of HIV infected cells by DNA-nanostructures that link T/NK cells to infected cells to kill these cells.

FIGS. 11A-11C. Schematic representations of possible methods to covalently link peptides or proteins directly to a DNA nanostructure. A. Conjugate DNA to the amino group on the surface of a peptide using a hetero-cross linker, sulfo SMCC. B. Click Chemistry. C. Amide bond formation.

FIG. 12. Schematic design of the DNA scaffolded adjuvant-antigen vaccine complex. The CpG ODN adjuvant molecules (FIG. 51) are depicted as curved purple ribbons in the model. The model antigen (streptavidin) is shown in red and the tetrahedral DNA scaffold is represented by green helices. The injected vaccine complexes bind specifically to B cells and non-specifically to dendritic cells and macrophages. The complexes are internalized by the three types of antigen-presenting cells, disassembled, and the individual peptide antigens are subsequently presented to T cells to activate antibody production by plasma B cells.

FIGS. 13A-13D. Antigen internalization in RAW 264.7 cells and primary DCs. (a) Representative flow cytometry result showing the cellular PE fluorescence in RAW 264.7 cells after 30 minute incubation with PE-STV and/or DNA scaffolds. (b) Representative confocal microscopy images showing internalization of PE-STV in RAW 264.7 cells. Index shows zoom-in images of representative cells. (c) Histogram showing time-dependent cellular internalization of PE-STV in RAW 264.7 cells. The mean fluorescent intensity of PE is plotted against the length of incubation time. Each column represents the average of three parallel measurements, and error bars are generated from the standard deviation. (d) Histogram showing the cellular internalization of PE-STV in primary DCs after 2 hour incubation. Each column represents the average of two parallel measurements and error bars are generated from standard error of the mean value.

FIGS. 14A-14B. Antibody response in BALB/c mice. (a) Immunization protocol. (b) Anti-STV IgG level after antigen challenge. The average antibody level was determined from the results of at least eight mice per group and is plotted here. The error bars are generated from the standard deviation. (c) Specific memory B cell response in mice assessed by ELISPOT. The average was calculated from results of at least eight mice per group and the asterisk indicates a p value of less than 0.05 as determined by an unpaired student t test.

FIGS. 15A-15B. Response against the double-stranded DNA scaffold. (a) Results analyzed by anti-dsDNA antibody ELISA kit. Relative OD indicates the ratio between the measured OD405 for each sample and that of a standard calibrator provided by the manufacture. (b) Confocal microscopy images assessing the anti-dsDNA antibody by ANA kit. i) and ii) slides incubated with positive and negative control serum provided by manufacture; iii) and iv) slides incubated with mouse serum from the Free CpG+STV group; v) and vi) slides incubated with mouse serum from Tetrahedron-CpG-STV group.

FIG. 16. Structure and sequence of the tetrahedral DNA nanostructure.

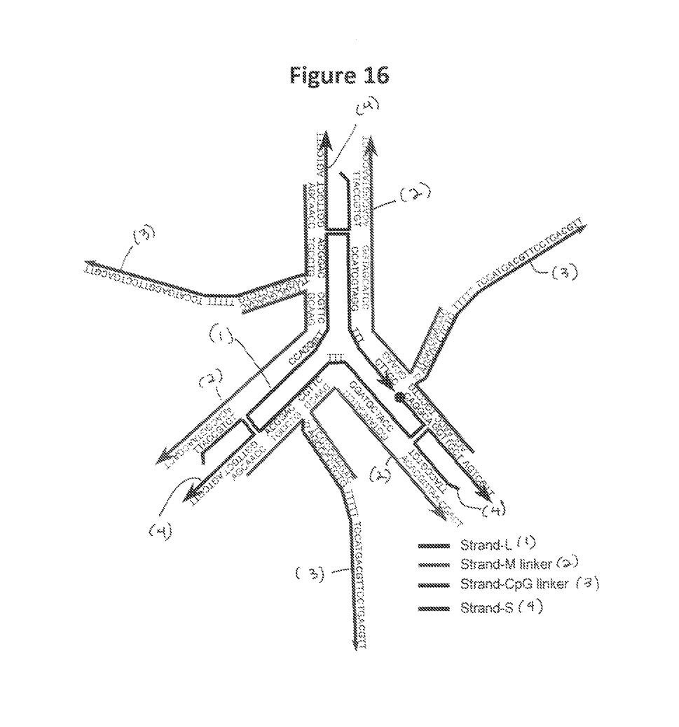

FIG. 17. Stability of the DNA scaffold in fetal bovine serum (FBS). Tetrahedral DNA structures were incubated with FBS at room temperature for 0.5, 1, 3, and 5 hours. The integrity of the DNA scaffolds was evaluated by non-denaturing agarose gel electrophoresis (1.2% agarose). A 100 bp DNA ladder is included in the far left lane in the gel.

FIG. 18. Antigen internalization in mouse B cell line A20. The internalization of directly linked ODN-STV in RAW 254.7 cells after 15 minute incubation is also plotted here for comparison. Antigen internalization in B cells is generally much weaker than in RAW 264.7 cells.

FIGS. 19A-19B. In vitro antigen internalization and in vivo antibody response induced by a branched DNA nanostructure. a, Antigen internalization of both DNA nanostructures in RAW 264.7 cells after 2 hour incubation. b, Antibody response in mice immunized with both DNA nanostructure-STV-CpG ODN complexes 13 days post antigen challenge. The average antibody level was calculated from the results of at least eight mice per group and is plotted here. The error bars are generated from the standard deviation. Stars indicate P values less than 0.05 according to a one-tailed unpaired student t test.

FIG. 20. Response against the tetrahedron-shaped DNA nanostructure. Results analyzed by ELISA. Relative OD indicates the ratio between the measured OD650 for each sample and that of the negative control provided by the manufacture of anti-dsDNA ELISA kit.

### DETAILED DESCRIPTION

A new synthetic way to construct vaccines, e.g. an HIV vaccine, is described herein. DNA-based virus-like particles, which can be tuned to function as effective vaccines against pathological conditions can be designed and assembled by combining 3D protein modeling, glycan and peptide grafting, novel addressable DNA-nanoscaffolds and rapid assessment of immune responses. This vaccine platform may induce a long-term production of multiple clones of high affinity neutralizing and/or inhibitory antibodies (e.g. anti-HIV antibodies). This novel approach may be used for the vaccine development against pathological conditions, such as infectious agents.

### DNA-Nanostructures

The present technology utilizes DNA nanostructures as a synthetic platform for vaccine construction. Specifically, the DNA-nanostructures may be used as scaffolds to assemble various antigenic components.

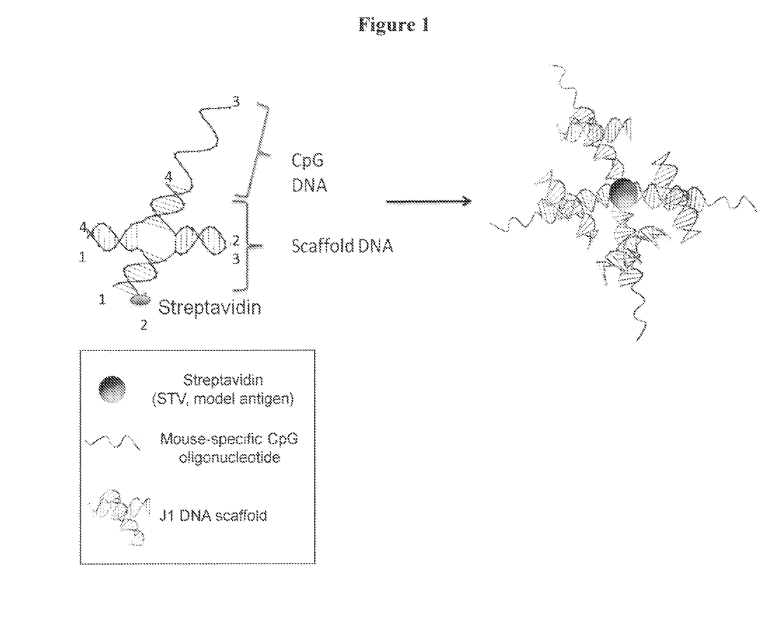

In certain embodiments, the DNA-nanostructures may be biotin-oligos, DNA-branches or DNA-tetrahedrons. These DNA-nanostructures may be prepared by methods known in the art. For example, oligonucleotides may be biotinylated using commercial labeling kits or may be purchased from commercial vendors (e.g. Integrated DNA Technologies). The DNA-branches are assembled based on the concept of base-pairing; no specific sequence is required; however, the sequences of each oligonucleotide must be partially complementary to certain other oligonucleotides to enable hybridization of all strands. For example, as shown in FIG. 1, four oligonucleotides with partial complementary sequences may be used to construct the DNA-branch. In certain embodiments one of the oligonucleotides is a CpG oligonucleotide. CpG oligodeoxynucleotides (CpG ODN) are short single stranded (ss) DNA that contain “C—P(phosphodiester or phosphorothioate)-G” structure. In certain embodiments, one of the oligonucleotides is biotinylated, which allows subsequent binding to an antigen, such as streptavidin. The DNA strands may be assembled by heating at 95° C. and then annealing at room temperature. In certain embodiments, the DNA-tetrahedrons may be prepared by methods described in Zhang, et al., Chem Commun, 46, 6792-6794 (2010) and He et al., Nature, 2008, 452, 198, which are herein incorporated by reference.

The length of each oligonucleotide or DNA strand is variable and depends on, for example, the type of nanostructure and the number of targeting moieties to be linked. In certain embodiments, the oligonucleotide or DNA strand is about 15 nucleotides in length to about 3000 nucleotides in length, such as 15 to 100 nucleotides, or 600-800 nucleotides.

For use in the present invention, the nucleic acids can be synthesized de novo using any of a number of procedures well known in the art. For example, the cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated oligonucleotide synthesizers available in the market.

### Targeting Moieties

In certain embodiments, at least one targeting moiety may be linked to the DNA-nanostructures. In certain embodiments, the composition comprises at least two targeting moieties (e.g. 2, 3, 4, 5, 6, 7, 8, 9, 10, etc.). In certain embodiments the targeting moieties are the same and in certain embodiments, the targeting moieties are different.

These targeting moieties may be assembled onto DNA-nanostructures at designated positions, i.e., in desired multi-valence, appropriate stoichiometry, and spatial orientations to elicit strong memory B cell responses. In certain embodiments, the targeting moieties are linked to the DNA nanostructure in polymeric forms. In certain embodiments, the polymeric form is trimeric.

In certain embodiments the targeting moiety is selected from the group consisting of antigens, aptamers, shRNAs and combinations thereof.

### Antigens

In certain embodiments the at least one targeting moiety is an antigen. As one skilled in the art will appreciate, it is not necessary to use the entire antigen. A selected portion of the antigen, for example the epitope, can be used.

As one skilled in the art will also appreciate, it is not necessary to use an antigen that is identical to a native antigen. The modified antigen can correspond essentially to the corresponding native antigen. As used herein “correspond essentially to” refers to an epitope that will elicit an immunological response at least substantially equivalent to the response generated by a native antigen. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest.

In certain embodiments the antigen is selected from the group consisting of a B-cell epitope, a T-cell epitope, a Tₕₑₗₚₑᵣ epitope, an HIV epitope, a neutralizing epitope, a gp120 epitope, a gp41 epitope, a glycan, a peptide, or a T-helper peptide. In certain embodiments, the antigen is streptavidin.

In certain embodiments, the antigen binds to a neutralizing antibody or an inhibitory antibody.

In certain embodiments, the neutralizing epitope is a peptide that mimics the CD4 binding site CD4b and binds to the neutralizing antibody b12.

In certain embodiments, the neutralizing epitope is a glycan that binds to the neutralizing antibody 2G12.

### Aptamers

In certain embodiments, the at least one targeting moiety is an aptamer.

Aptamers are single stranded oligonucleotides that can naturally fold into different 3-dimensional structures, which have the capability of binding specifically to biosurfaces, a target compound or a moiety. The term “conformational change” refers to the process by which a nucleic acid, such as an aptamer, adopts a different secondary or tertiary structure. The term “fold” may be substituted for conformational change.

Aptamers have low immunogenicity. They can easily be synthesized in large quantities at a relatively low cost and are amendable to a variety of chemical modifications that confer both resistance to degradation and improved pharmacokinetics in vivo. The smaller size of aptamers compared with that of antibodies (<15 kDa versus 150 kDa) facilitates their in vivo delivery by promoting better tissue penetration.

Aptamers have advantages over more traditional affinity molecules such as antibodies in that they are very stable, can be easily synthesized, and can be chemically manipulated with relative ease. Aptamer synthesis is potentially far cheaper and reproducible than antibody-based diagnostic tests. Aptamers are produced by solid phase chemical synthesis, an accurate and reproducible process with consistency among production batches. An aptamer can be produced in large quantities by polymerase chain reaction (PCR) and once the sequence is known, can be assembled from individual naturally occurring nucleotides and/or synthetic nucleotides. Aptamers are stable to long-term storage at room temperature, and, if denatured, aptamers can easily be renatured, a feature not shared by antibodies. Furthermore, aptamers have the potential to measure concentrations of ligand in orders of magnitude lower (parts per trillion or even quadrillion) than those antibody-based diagnostic tests. These characteristics of aptamers make them attractive for diagnostic applications.

Aptamers are typically oligonucleotides that may be single stranded oligodeoxynucleotides, oligoribonucleotides, or modified oligodeoxynucleotide or oligoribonucleotides. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6-diaminopurine; methylpsuedouracil; 1-methylguanine; 1-methylcytosine.

Aptamers may be synthesized using conventional phosphodiester linked nucleotides and synthesized using standard solid or solution phase synthesis techniques, which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl(1-12C) and R6 is alkyl(1-9C) is joined to adjacent nucleotides through —O— or —S—.

In certain embodiments, modifications are made to the aptamer(s). Additional modifications to the aptamer include 2′O-methyl modification of the pyrimidines. In other embodiments, all of the nucleotides in the aptamer are 2′O-methyl modified. Alternatively, the pyrimidines, or all the nucleotides, may be modified with 2′fluoros (both pyrimidines and purines). Additional modifications to the nucleotides in the aptamer include large molecular weight conjugates like pegylation, lipid-based modifications (e.g., cholesterol) or nanoparticles (e.g., PEI or chitosan) to improve the pharmacokinetic/dynamic profile of the chimera.

In certain embodiments, modifications are introduced into the stem sequence in the aptamer. Different nucleotides can be used as long as the structure of the stem is retained.

In certain embodiments, the aptamer molecule is about 10 nucleotides in length to about 1,000 nucleotides in length. In certain embodiments, the aptamer molecule is not more than 500 nucleotides in length. In certain embodiments, the aptamer molecule is not more than 100 nucleotides in length. In certain embodiments, the total scaffold of the aptamer is about 80 nucleotides. In certain embodiments, the binding region is about 20-60 nucleotides, such as about 40 nucleotides.

In certain embodiments, the aptamer binds to an HIV epitope, or a cell surface receptor expressed on an immune cell (e.g., T-cell, NK-cell, etc.). In certain embodiments, the HIV epitope is a gp120 epitope. In certain embodiments, the cell surface receptor is CD16 or cytotoxic T-lymphocyte antigen 4 (CTLA4).

### RNAi Molecules

In certain embodiments, the at least one targeting moiety is an RNA interference (RNAi) molecule. In certain embodiments, the RNAi molecule is shRNA, siRNA or miRNA.

A small hairpin RNA or short hairpin RNA (shRNA) is a sequence of RNA that makes a tight hairpin turn that can be used to silence gene expression via RNA interference.

In certain embodiments, the shRNA is specific for FoxoP3.

### Detection Means

In certain embodiments, the composition further comprises a detection means. In certain embodiments, the detection means is linked to the DNA nanostructure.

In certain embodiments, the targeting moiety may comprise a detection means. In certain embodiments, the targeting moiety is operably linked to the detection means.

A number of “molecular beacons” (such as fluorescence compounds) can be attached to the DNA nanostructure or targeting moiety to provide a means for signaling the presence of and quantifying a target chemical, cell or biological agent, for example, R-Phycoerythrin (PE). Other exemplary detection labels that could be attached to the targeting moiety include biotin, any fluorescent dye, amine modification, horseradish peroxidase, alkaline phosphatase, etc. In certain embodiments, the detection means is linked to the DNA nanostructure, and in certain embodiments, the detection means is linked to the targeting moiety.

### CpG Oligonucleotides and Other Adjuvants

In certain embodiments, the composition further comprises at least one adjuvant. In certain embodiments, the adjuvant is linked to the DNA nanostructure. In certain embodiments, the composition further comprises at least two adjuvants (e.g. 2, 3, 4, 5, 6, 7, 8, 9, 10, etc.). In certain embodiments, the adjuvants are the same and in certain embodiments, the adjuvants are different. In certain embodiments, all of the adjuvants are linked to the DNA-nanostructure. In certain embodiments, none of the adjuvants are linked to the DNA-nanostructure. In certain embodiments, one or more of the adjuvants are linked to the DNA-nanostructure and one or more of the adjuvants are not linked to the DNA-nanostructure. When an adjuvant(s) is not linked to the DNA-nanostructure, the composition can be administered before, after, and/or simultaneously with the adjuvant(s).

A conventional “adjuvant” is any molecule or compound that nonspecifically stimulates the humoral and/or cellular immune response. They are considered to be nonspecific because they only produce an immune response in the presence of an antigen. Adjuvants allow much smaller doses of antigen to be used and are essential to inducing a strong antibody response to soluble antigens.

Immunostimulatory oligonucleotides, which directly activate lymphocytes and co-stimulate an antigen-specific response, are fundamentally different from conventional adjuvants (e.g., aluminum precipitates), which are inert when injected alone and are thought to work through absorbing the antigen and thereby presenting it more effectively to immune cells.

In certain embodiments, an adjuvant may be an oligonucleotide containing at least one immunostimulatory CpG motif Additional suitable adjuvants include but are not limited to surfactants, e.g., hexadecylamine, octadecylamine, lysolecithin, dimethyldioctadecylammonium bromide, N,N-dioctadecyl-N′—N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, aimethylglycine, tuftsin, oil emulsions, aluminum (alum), aluminum hydroxide, incomplete Freud's adjuvant, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). CpG

### Oligonucleotides

An oligonucleotide containing at least one immunostimulatory CpG motif can be used to activate the immune response. CpG DNA for use as a vaccine adjuvant is known in the art and described, for example, in Bode et al., Expert. Rev. Vaccines, 10(4), 499-511 (2011) and U.S. Publication 2008-0124366, which are incorporated herein by reference.

As used herein the article “a” or “an” is used to mean “one or more.” For example “an oligonucleotide” would mean “one or more oligonucleotide.”

The term “nucleic acid” or “oligonucleotide” refers to a polymeric form of nucleotides at least five bases in length. The term “oligonucleotide” includes both single and double-stranded forms of nucleic acid. The nucleotides of the invention can be deoxyribonucleotides, ribonucleotides, or modified forms of either nucleotide. Generally, double-stranded molecules are more stable in vivo, although single-stranded molecules have increased activity when they contain a synthetic backbone.

An “oligodeoxyribonucleotide” (ODN) as used herein is a deoxyribonucleic acid sequence from about 3-1000 (or any integer in between) bases in length. In certain embodiments, the ODN is about 3 to about 50 bases in length. Lymphocyte ODN uptake is regulated by cell activation. For example, B-cells that take up CpG ODNs proliferate and secrete increased amounts of immunoglobulin. Certain oligonucleotides containing at least one unmethylated cytosine-guanine (CpG) dinucleotide activate the immune response.

A “CpG” or “CpG motif” refers to a nucleic acid having a cytosine followed by a guanine linked by a phosphate bond. The term “methylated CpG” refers to the methylation of the cytosine on the pyrimidine ring, usually occurring at the 5-position of the pyrimidine ring. The term “unmethylated CpG” refers to the absence of methylation of the cytosine on the pyrimidine ring. Methylation, partial removal, or removal of an unmethylated CpG motif in an oligonucleotide of the invention is believed to reduce its effect. Methylation or removal of all unmethylated CpG motifs in an oligonucleotide substantially reduces its effect. The effect of methylation or removal of a CpG motif is “substantial” if the effect is similar to that of an oligonucleotide that does not contain a CpG motif.

In certain embodiments the CpG oligonucleotide is in the range of about 8 to 30 bases in size, or about 15 to 20 bases in size. For use in the present invention, the nucleic acids can be synthesized de novo using any of a number of procedures well known in the art. For example, the cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated oligonucleotide synthesizers available in the market.

As used herein the term “palindromic sequence” means an inverted repeat (i.e., a sequence such as ABCDEE′D′C′B′A′ in which A and A′ are bases capable of forming the usual Watson-Crick base pairs. In vivo, such sequences may form double-stranded structures.

A “stabilized nucleic acid molecule” shall mean a nucleic acid molecule that is relatively resistant to in vivo degradation (e.g., via an exo- or endo-nuclease). Stabilization can be a function of length or secondary structure. Unmethylated CpG containing nucleic acid molecules that are tens to hundreds of kilobases long are relatively resistant to in vivo degradation. For shorter immunostimulatory nucleic acid molecules, secondary structure can stabilize and increase their effect. For example, if the 3′ end of a nucleic acid molecule has self-complementarity to an upstream region, so that it can fold back and form a sort of stem loop structure, then the nucleic acid molecule becomes stabilized and therefore exhibits more activity.

In certain embodiments, stabilized nucleic acid molecules of the instant invention have a modified backbone. It has been shown that modification of the oligonucleotide backbone provides enhanced activity of the CpG molecules of the invention when administered in vivo. CpG constructs, including at least two phosphorothioate linkages at the 5′ end of the oligodeoxyribonucleotide and multiple phosphorothioate linkages at the 3′ end, provided maximal activity and protected the oligodeoxyribonucleotide from degradation by intracellular exo- and endo-nucleases. Other modified oligodeoxyribonucleotides include phosphodiester modified oligodeoxyribonucleotide, combinations of phosphodiester, phosphorodithioate, and phosphorothioate oligodeoxyribonucleotide, methylphosphonate, methylphosphorothioate, phosphorodithioate, or methylphosphorothioate and combinations thereof. The phosphate backbone modification can occur at the 5′ end of the nucleic acid, for example at the first two nucleotides of the 5′ end of the nucleic acid. The phosphate backbone modification may occur at the 3′ end of the nucleic acid, for example at the last five nucleotides of the 3′ end of the nucleic acid. Nontraditional bases such as inosine and queosine, as well as acetyl-, thio- and similarly modified forms of adenine, cytidine, guanine, thymine, and uridine can also be included, which are not as easily recognized by endogenous endonucleases. Other stabilized nucleic acid molecules include: nonionic DNA analogs, such as alkyl- and aryl-phosphonates (in which the charged oxygen moiety is alkylated). Nucleic acid molecules that contain a diol, such as tetrahyleneglycol or hexaethyleneglycol, at either or both termini are also included.

DNA containing unmethylated CpG dinucleotide motifs in the context of certain flanking sequences has been found to be a potent stimulator of several types of immune cells in vitro. (Ballas, et al., J. Immunol. 157:1840 (1996); Cowdrey, et al., J. Immunol. 156:4570 (1996); Krieg, et al., Nature 374:546 (1995)) Depending on the flanking sequences, certain CpG motifs may be more immunostimulatory for B cell or T cell responses, and preferentially stimulate certain species. When a humoral response is desired, preferred immunostimulatory oligonucleotides comprising an unmethylated CpG motif will be those that preferentially stimulate a B cell response. When cell-mediated immunity is desired, preferred immunostimulatory oligonucleotides comprising at least one unmethylated CpG dinucleotide will be those that stimulate secretion of cytokines known to facilitate a CD8+ T cell response.

The immunostimulatory oligonucleotides of the invention may be chemically modified in a number of ways in order to stabilize the oligonucleotide against endogenous endonucleases. As used herein, these contain “synthetic phosphodiester backbones.” For example, the oligonucleotides may contain other than phosphodiester linkages in which the nucleotides at the 5′ end and/or 3′ end of the oligonucleotide have been replaced with any number of nontraditional bases or chemical groups, such as phosphorothioate-modified nucleotides. The immunostimulatory oligonucleotide comprising at least one unmethylated CpG dinucleotide may preferably be modified with at least one such phosphorothioate-modified nucleotide. Oligonucleotides with phosphorothioate-modified linkages may be prepared using methods well known in the field such as phosphoramidite (Agrawal, et al., Proc. Natl. Acad. Sci. 85:7079 (1988)) or H-phosphonate (Froehler, et al., Tetrahedron Lett. 27:5575 (1986)). Examples of other modifying chemical groups include alkylphosphonates, phosphorodithioates, alkylphosphorothioates, phosphoramidates, 2-O-methyls, carbamates, acetamidates, carboxymethyl esters, carbonates, and phosphate triesters. Oligonucleotides with these linkages can be prepared according to known methods (Goodchild, Chem. Rev. 90:543 (1990); Uhlmann, et al., Chem. Rev. 90:534 (1990); and Agrawal, et al., Trends Biotechnol. 10:152 (1992)). A “partially synthetic backbone” is a backbone where some of the oligonucleotides are modified, and a “completely synthetic backbone” is one where all of the oligonucleotides are modified. A “natural phosphodiester backbone” is one where the oligonucleotides have not been modified.

Other stabilized nucleic acid molecules include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acid molecules which contain diol, such as tetraethyleneglycol or hexaethyleneglycol, at either or both termini have also been shown to be substantially resistant to nuclease degradation.

A “subject” shall mean a human or vertebrate animal including a dog, cat, horse, cow, pig, sheep, goat, chicken, monkey, rat, and mouse. Nucleic acids containing an unmethylated CpG can be effective in any mammal, such as a human. Different nucleic acids containing an unmethylated CpG can cause optimal immune stimulation depending on the mammalian species. Thus an oligonucleotide causing optimal stimulation in humans may not cause optimal stimulation in a mouse. One of skill in the art can identify the optimal oligonucleotides useful for a particular mammalian species of interest.

The stimulation index of a particular immunostimulatory CpG ODN to effect an immune response can be tested in various immune cell assays. The stimulation index of the immune response can be assayed by measuring various immune parameters, e.g., measuring the antibody-forming capacity, number of lymphocyte subpopulations, mixed leukocyte response assay, lymphocyte proliferation assay. The stimulation of the immune response can also be measured in an assay to determine resistance to infection or tumor growth. Methods for measuring a stimulation index are well known to one of skill in the art. For example, one assay is the incorporation of ³H thymidine in a murine B cell culture, which has been contacted with a 20 pM of oligonucleotide for 20 h at 37° C. and has been pulsed with 1 pCi of ³H uridine; and harvested and counted 4 h later. The induction of secretion of a particular cytokine can also be used to assess the stimulation index. In one method, the stimulation index of the CpG ODN with regard to B-cell proliferation is at least about 5, at least about 10, at least about 15, or even at least about 20 (as described in detail in U.S. Pat. No. 6,239,116), while recognizing that there are differences in the stimulation index among individuals.

The term “polynucleotide” or “nucleic acid sequence” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The nucleotides of the invention can be ribonucleotides, deoxyribonucleotides, or modified forms of either nucleotide. The term includes single and double stranded forms of DNA.

### Methods for Making Immunostimulatory Nucleic Acids

For use in the instant invention, nucleic acids can be synthesized de novo using any of a number of procedures well known in the art. For example, the B-cyanoethyl phosphoramidite method (S. L. Beaucage and M. H. Caruthers, 1981, Tet. Let. 22:1859); nucleoside H-phosphonate method (Garegg, et al., 1986, Tet. Let. 27:4051-4051; Froehler, et al., 1986, Nucl. Acid. Res. 14:5399-5407; Garegg, et al., 1986, Tet. Let. 27:4055-4058, Gaffney, et al., 1988), Tet. Let. 29:2619-2622. These chemistries can be performed by a variety of automated oligonucleotide synthesizers available in the market. Alternatively, oligonucleotides can be prepared from existing nucleic acid sequences (e.g., genomic or cDNA) using known techniques, such as those employing restriction enzymes, exonucleases or endonucleases.

For use in vivo, nucleic acids are preferably relatively resistant to degradation (e.g., via endo- and exo-nucleases). Secondary structures, such as stem loops, can stabilize nucleic acids against degradation. Alternatively, nucleic acid stabilization can be accomplished via phosphate backbone modifications. A stabilized nucleic acid can be accomplished via phosphate backbone modifications. A stabilized nucleic acid has at least a partial phosphorothioate modified backbone. Phosphorothioates may be synthesized using automated techniques employing either phosphoramidate or H-phosphonate chemistries. Aryl- and alkyl-phosphonates can be made for example as described in U.S. Pat. No. 4,469,863; and allcylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., 1990, Chem Rev. 90:544; Goodchild, J., 1990, Bioconjugate Chem. 1:165). 2′-O-methyl nucleic acids with CpG motifs also cause immune activation, as do ethoxy-modified CpG nucleic acids. In fact, no backbone modifications have been found that completely abolish the CpG effect, although it is greatly reduced by replacing the C with a 5-methyl C.

Linking the DNA Nanostructure with the at Least One Targeting Moiety and/or Adjuvant.

Chemistries that can be used to link the at least one targeting moiety and/or adjuvant to the DNA nanostructure are known in the art, such as disulfide linkages, amino linkages, covalent linkages, etc. Additional linkages and modifications can be found on the world-wide-web at trilinkbiotech.com/products/oligo/oligo_modifications.asp.

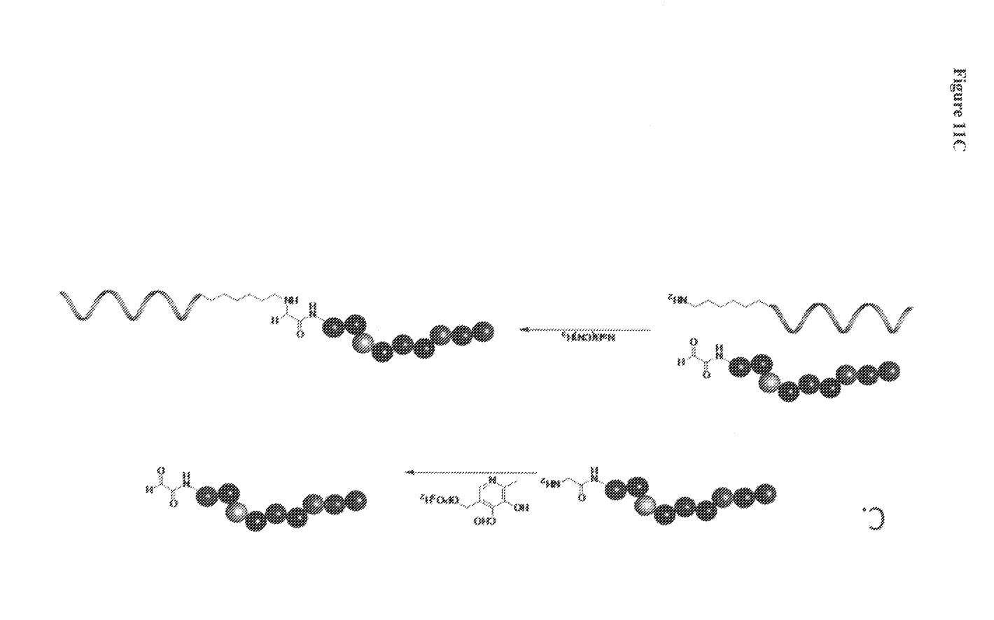

In certain embodiments, “linked” includes directly linking (covalently or non-covalently binding) the at least one targeting moiety and/or adjuvant to the DNA nanostructure. In certain embodiments, a direct linkage maybe made covalently. For example, as described in FIG. 11, the covalent linkage may be made by conjugating the DNA to an amino group on the surface of a peptide using a hetero-cross linker, sulfo SMCC, through Click chemistry, or through the formation of an amide bond. Click chemistry is a two-step process known in the art that uses quantitative chemical reactions of alkyne and azide moieties to create covalent carbon-heteroatom bonds between biochemical species (Rostovtsev, et al., Angew Chem. Int. Ed. Engl., 2002, 41(12): 2596-9). The reaction uses copper(I) as a catalyst and forms a 1,2,3-triazole between an azide and terminal alkyne (Moses et al., Chem. Soc. Rev. 2007, 36(8):1249-62).

In certain embodiments, “linked” includes linking the at least one targeting moiety and/or adjuvant to the DNA nanostructure using a linker, e.g., a nucleotide linker, e.g., the nucleotide sequence “AA” or “TT” or “UU”.

In certain embodiments, the linker is a binding pair. In certain embodiments, the “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate, IgG-protein A, antigen-antibody, and the like. In certain embodiments, a first member of the binding pair comprises avidin or streptavidin and a second member of the binding pair comprises biotin.

As used herein the terms “link”, “conjugate” and “engraft” may be used interchangeably.

### Nanovaccines

In certain embodiments, compositions described herein are “nanovaccines”. The term “nanovaccine” refers to a composition capable of producing an immune response. In certain embodiments, the composition of the present invention may be used in the prophylactic or therapeutic treatment of a pathological condition. In certain embodiments, the pathological condition is a disease, for example, HIV or cancer. In certain embodiments, a nanovaccine composition, according to the invention, would produce immunity against disease in individuals. In certain embodiments, the pathological condition is substance abuse or addiction.

In certain embodiments the nanovaccine is about 20-200 nm, such as 50-100 nm in size.

### HIV Nanovaccine

In certain embodiments, the composition of the present invention may be used in the prophylactic or therapeutic treatment of HIV (active or latent infections).

In certain embodiments, the composition comprises a DNA-nanostructure and at least two targeting moieties (e.g. 2, 3, 4, 5, 6, 7, 8, 9, 10, etc.), wherein the targeting moieties are linked to the DNA-nanostructure. In certain embodiments, the targeting moieties are the same and in certain embodiments the targeting moieties are different.

In certain embodiments the targeting moieties are HW neutralizing epitopes. In certain embodiments, the neutralizing epitopes are gp120 epitopes, gp4b epitopes or CD4b epitopes. In certain embodiments, one neutralizing epitope is a glycan that binds to the neutralizing monoclonal antibody 2G12 and the other neutralizing epitope is a CD4b peptide that binds to the neutralizing monoclonal antibody b12. In certain embodiments, the glycan and peptide are linked to the DNA-nanostructure at positions, distances and configurations to mimic trimetric CD4bs or desired glycan structures.

In certain embodiments, the composition further comprises at least one T helper-peptide and at least one adjuvant. In certain embodiments the adjuvant is an oligonucleotide containing at least one immunostimulatory CpG motif. In certain embodiments, the T helper-peptide and the adjuvant are linked to the DNA-nanostructure at designated positions apart from the neutralizing epitopes.

In certain embodiments, the composition further comprises additional neutralizing epitopes.

In certain embodiments, the composition resembles viral like particles and recruits gp120-specific B cells, T helper cells and dendritic cells to the same microenvironment for their interactions and subsequent activation. These targeting moieties may be assembled onto DNA-nanostructures at designated positions, i.e., in desired multi-valence, appropriate stoichiometry, and spatial orientations to elicit strong memory B cell responses.

In certain embodiments, the composition elicits neutralizing and/or inhibitory antibody responses.

### Bi-Specific-DNA-Nanostructures

In certain embodiments, the composition of the present invention may be used in the prophylactic or therapeutic treatment of a pathological condition. In certain embodiments, the pathological condition is a disease, for example, HIV or cancer. In certain embodiments, the composition of the present invention may be used to modulate immune responses.

### HIV Bi-Specific-DNA-Nanostructures

In certain embodiments, the composition of the present invention may be used in the prophylactic or therapeutic treatment of HIV (active or latent infections).

In certain embodiments, the composition comprises a DNA-nanostructure and at least two targeting moieties (e.g. 2, 3, 4, 5, 6, 7, 8, 9, 10, etc.), wherein the targeting moieties are linked to the DNA-nanostructure.

In certain embodiments, the targeting moieties are a first aptamer and a second aptamer, wherein the first and second aptamers are different. In certain embodiments, first aptamer binds to an HW infected cell. In certain embodiments, the first aptamer binds to an HIV epitope (e.g. gp120 binding aptamer). In certain embodiments, the second aptamer binds to an immune cell, for example, by binding to a cell surface receptor expressed on the immune cell (e.g. T-cell or NK-cell). In certain embodiments, the second aptamer binds to CD 16. In certain embodiments, the targeting moiety is a peptide or a sugar ligand.

In certain embodiments, the composition may be used to treat latent HW infections. In certain embodiments, the composition further comprises activation agents for T cells or macrophages that serve as an HW reservoir. In certain embodiments, the activation agent is a cytokine activator. In certain embodiments, the agent is TNF-alpha, or is able to activate TNF-alpha

In certain embodiments, the composition engages the immune cell to attack the HIV-infected cells.

### Combination Therapy

In certain embodiments, a nanovaccine composition as described herein may be used in combination with a bi-specific-DNA-nanostructure composition as described herein for the prophylactic or therapeutic treatment of a pathological condition. In certain embodiments, the pathological condition is a disease, for example, cancer.

In certain embodiments, the bi-specific-DNA-nanostructure compositions engage immune cells to attack cancer cells while the nanovaccine compositions induce tumor immunity.

In certain embodiments, the bi-specific-DNA-nanostructure composition further comprises a sensor, wherein the sensor is linked to the DNA-nanostructure. In certain embodiments, the sensor is an oligonucleotide. In certain embodiments, the nanovaccine composition further comprises a detector, wherein the detector is linked to the DNA-nanostructure. In certain embodiments, the detector is an oligonucleotide. In certain embodiments, the sensor of the bi-specific-DNA-nanostructure composition binds to the detector of the nanovaccine composition (e.g. the sensor and the detector may hybridize through complementary sequences).

### Antibodies and Methods of Making Antibodies

Polyclonal and monoclonal antibodies, which recognize compositions described herein, can be prepared and analyzed by methods known to those skilled in the art. For example, the antibodies can be prepared by the methods described below. These antibodies may be capable of passively protecting a mammal from a pathological condition.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J. Mol. Biol. 222:581-597, 1991). The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

Polyclonal and monoclonal antibodies can be prepared by methods known to those skilled in the art.

Compositions to be used for the immunization of animals and the subsequent preparation of antibodies are described herein. In certain embodiments the animal is a mammal, such as a mouse, rat, hamster, guinea pig, horse, monkey, rabbit, goat, and sheep. This immunization can be performed by any existing method, including typically used intravenous injections, subcutaneous injections, and intraperitoneal injections. There are no restrictions as to the immunization intervals. Immunization may be carried out at intervals of several days to several weeks, preferably four to 21 days. A mouse can be immunized, for example, at a single dose of 10 to 100 μg (for example, 20 to 40 μg) of the composition, but the dose is not limited to these values. In certain embodiments, the animal will be given multiple doses, such as three. In one embodiment, the animal is given three doses of 10 μg.

In another embodiment, antibodies or antibody fragments can be isolated from an antibody phage library, produced by using the technique reported by McCafferty et al. (Nature 348:552-554 (1990)). Clackson et al. (Nature 352:624-628 (1991)) and Marks et al. (J. Mol. Biol. 222:581-597 (1991)) reported on the respective isolation of mouse and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal antibodies, instead of using conventional hybridoma technology for monoclonal antibody production.

The antibodies of the present invention are antibodies that provide passive immunity to a pathological condition.

The antibodies of the present invention described above can be used in a passive immunity treatment of an individual that has a pathological condition.

### Diagnostic & Therapeutic Uses

In the methods of the present invention, the subject may be a vertebrate animal including a human, dog, cat, horse, cow, pig, sheep, goat, chicken, monkey, rat, or mouse.

In one embodiment, the invention provides a method for stimulating an immune response in a subject by administering a therapeutically effective amount of composition as described herein. This invention provides administering to a subject having or at risk of having a pathological condition, a therapeutically effective dose of a pharmaceutical composition described herein and a pharmaceutically acceptable carrier. “Administering” the pharmaceutical composition of the present invention may be accomplished as described below and by any means known to the skilled artisan.

### Formulations and Methods of Administration

The compositions of the invention may be formulated as pharmaceutical composition and administered to a subject, such as a human patient, in a variety of forms adapted to the chosen route of administration, i.e., orally, mucosally, intranasally, intradermally, intratumorally or parenterally, by intravenous, intramuscular, topical or subcutaneous routes.

Formulations will contain an effective amount of the active ingredient in a vehicle, the effective amount being readily determined by one skilled in the art. “Effective amount” is meant to indicate the quantity of a compound necessary or sufficient to realize a desired biologic effect. For example, an effective amount of a composition described herein could be the amount necessary to prevent, to cure or at least partially arrest symptoms and complications. The active ingredient may typically range from about 1% to about 95% (w/w) of the composition, or even higher or lower if appropriate. The amount for any particular application can vary depending on such factors as the disease or condition being treated, the particular composition being administered (e.g., specific combination of DNA nanostructure and targeting moieties), or the severity of the condition. The quantity to be administered depends upon factors such as the age, weight and physical condition of the animal or the human subject considered for vaccination, kind of concurrent treatment, if any, and nature of the antigen administered. The quantity also depends upon the capacity of the animal's immune system to synthesize antibodies, and the degree of protection desired. Typically, dosages used in vitro may provide useful guidance in the amounts useful for in situ administration of the composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Reminpton's Pharmaceutical Sciences, 17th ed., Mack Publishing Co., Easton, Pa., 1990, each of which is herein incorporated by reference. Additionally, effective dosages can be readily established by one of ordinary skill in the art through routine trials establishing dose response curves. The subject is immunized by administration of the composition thereof in one or more doses. Multiple doses may be administered as is required to maintain a state of immunity to the target. For example, the initial dose may be followed up with a booster dosage after a period of about four weeks to enhance the immunogenic response. Further booster dosages may also be administered. The composition may be administered multiple (e.g., 2, 3, 4 or 5) times at an interval of, e.g., about 1, 2, 3, 4, 5, 6 or 7, 14, or 21 days apart.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the present compositions may be combined with one or more excipients and used in the form of ingestible tablets, buccal tablets, troches, capsules, elixirs, suspensions, syrups, wafers, and the like. Such preparations should contain at least 0.1% of the present composition. The percentage of the compositions may, of course, be varied and may conveniently be between about 2 to about 60% of the weight of a given unit dosage form. The amount of present composition in such therapeutically useful preparations is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the present composition, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the present compositions may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the present composition that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating a composition described herein in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compositions described herein may be applied in pure form, i.e., when they are liquids. However, it will generally be desirable to administer them to the skin as formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compositions of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compositions described herein required for use in treatment will vary with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### General Terminology

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

“Synthetic” aptamers are those prepared by chemical synthesis. The aptamers may also be produced by recombinant nucleic acid methods.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

By “fragment” or “portion” is meant a full length or less than full length of the nucleotide sequence.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched nucleic acid. Specificity is typically the function of post-hybridization washes, the critical factors being the ionic strength and temperature of the final wash solution. For DNA-DNA hybrids, the Tₘ can be approximated from the equation of Meinkoth and Wahl: Tₘ 81.5° C.+16.6 (log M)+0.41 (% GC)−0.61 (% form)−500/L. M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule containing less than 31 sequential nucleotides that is transcribed into an RNAi molecule. Such an isolated RNAi molecule may, for example, form a hairpin structure with a duplex 21 base pairs in length that is complementary or hybridizes to a sequence in a gene of interest, and remains stably bound under stringent conditions (as defined by methods well known in the art, e.g., in Sambrook and Russell, 2001). Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

In certain embodiments a DNA sequence may encode a siRNA, as well as double-stranded interfering RNA molecules, which are also useful to inhibit expression of a target gene.

The terms “protein,” “peptide” and “polypeptide” are used interchangeably herein. As used herein, the terms “a” or “an” are used to mean “one or more.”

“Recombinant DNA molecule” is a combination of DNA sequences that are joined together using recombinant DNA technology and procedures used to join together DNA sequences as described, for example, in Sambrook and Russell (2001).

“Operably-linked” nucleic acids refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The term “amino acid” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in Dextrorotary or Levorotary stereoisomeric forms, as well as unnatural amino acids (e.g., phosphoserine, phosphothreonine, phosphotyrosine, hydroxyproline, and gamma-carboxyglutamate; hippuric acid, octahydroindole-2-carboxylic acid, statine, 1,2,3,4-tetrahydroisoquinoline-3-carboxylic acid, penicillamine, ornithine, citruline, alpha-methyl-alanine, para-benzoylphenylalanine, phenylglycine, propargylglycine, sarcosine, and tert-butylglycine). The term also comprises natural and unnatural amino acids (Dextrorotary and Levorotary stereoisomers) bearing a conventional amino protecting group (e.g. acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc, and documents cited therein). An amino acid can be linked to the remainder of a compound of formula (I) through the carboxy terminus, the amino terminus, or through any other convenient point of attachment, such as, for example, through the sulfur of cysteine.

The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound through the carboxy terminus, the amino terminus, or through any other convenient point of attachment, such as, for example, through the sulfur of a cysteine. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right.

The invention will now be illustrated by the following non-limiting Examples.

### Example 1

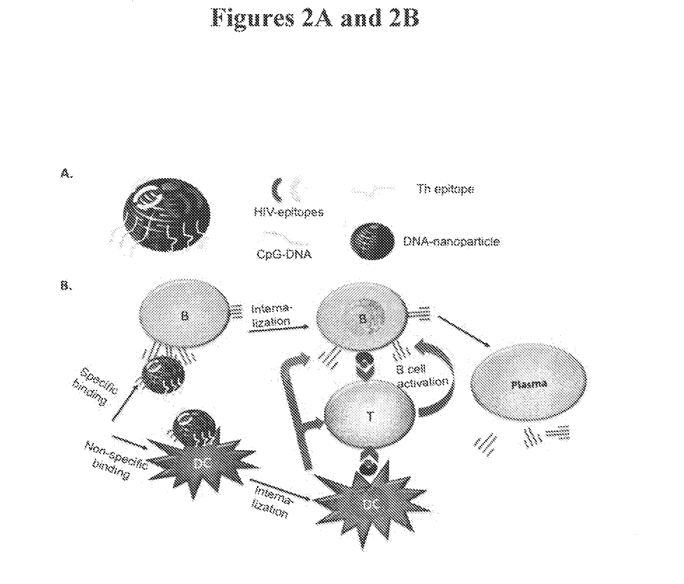

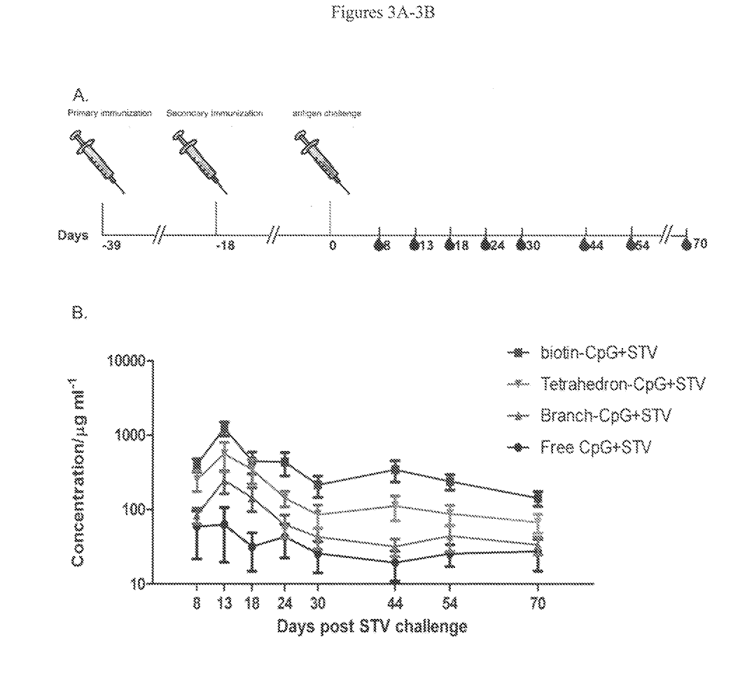

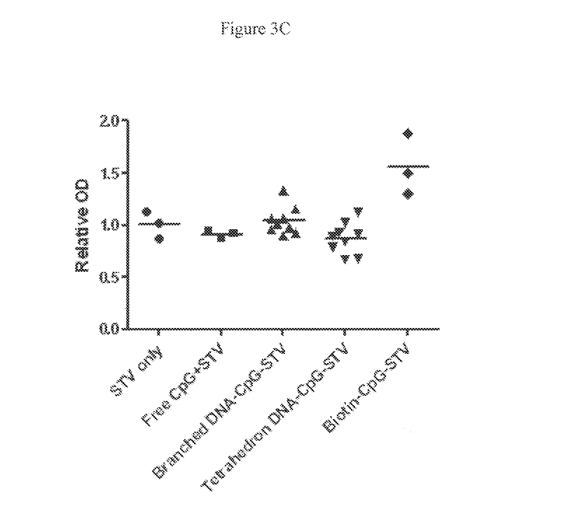

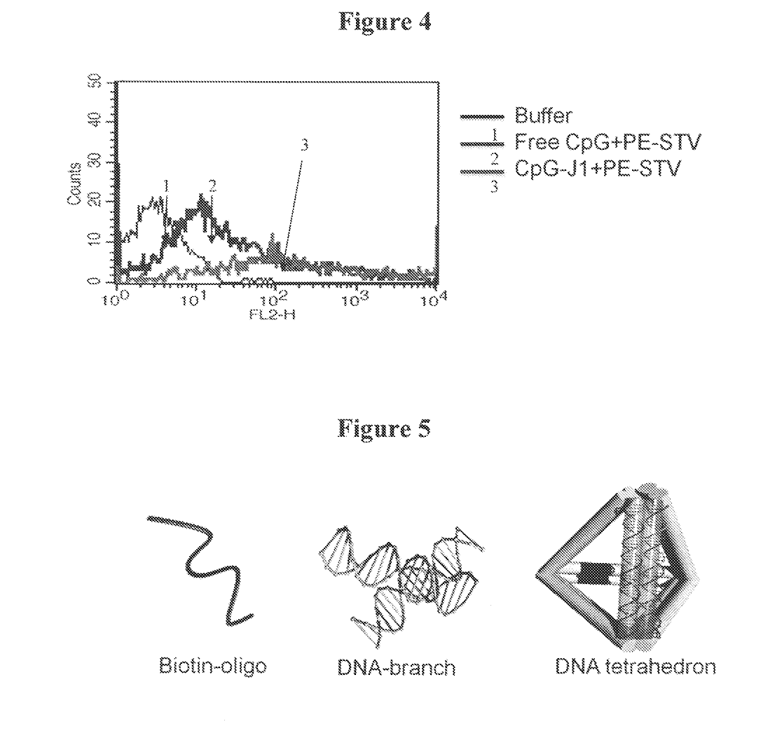

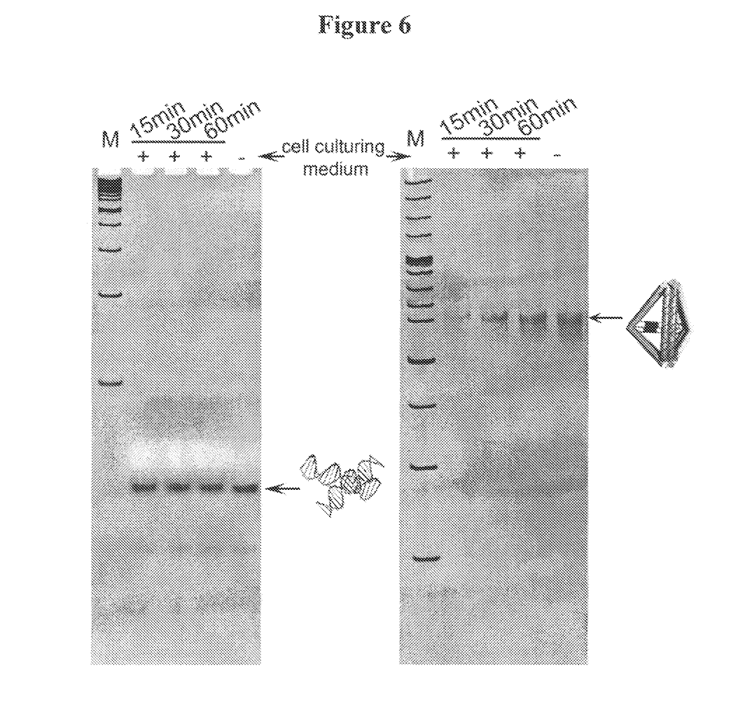

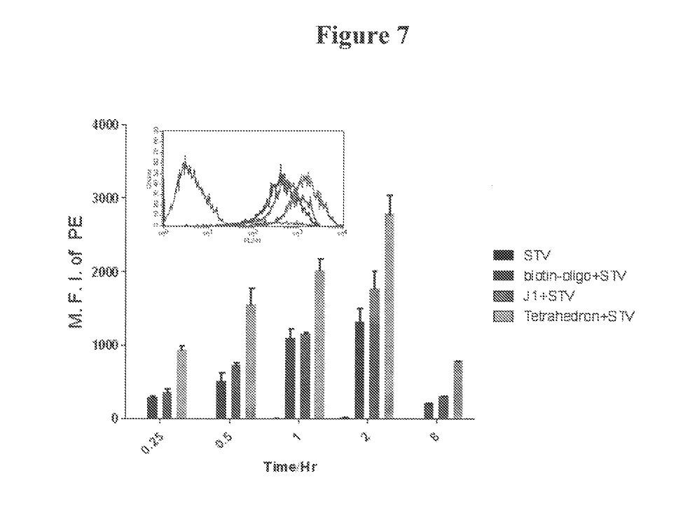

Safe and effective vaccines offer the best health intervention in disease control. However, current strategies for vaccine development suffer from either safety or ineffective issues. Accordingly, DNA nanostructures as scaffolds to assemble various antigenic components have been explored and are described herein. A proof-of-concept immunogenicity test was conducted by assembling a model antigen (streptavidin) and immunoadjuvant CpG oligonucleotide onto a DNA-branch nanostructure (FIG. 1). A schematic illustrating an immune response cascade elicited by this assembly is depicted in FIG. 2. Antibody responses against the DNA nanostructure antigen were evaluated in mice, as shown in FIG. 3. The antigen engineered onto the DNA-scaffolds elicited stronger memory antibody responses than the one induced by the same antigen in the conventional way. Additionally, conjugated CpG (CpG-J1+PE-STV) showed higher cellular uptake in vivo as compared to free CpG (free CpG+PE-STV) (FIG. 4). As shown in FIG. 5, the DNA-nanostructures may be made using multiple types of scaffolds, for example, such as biotin oligos, DNA-branches (e.g. J1 described above) and DNA-tetrahedrons. The DNA-nanostructures with different scaffolds may be compared using a variety of in vitro and in vivo tests to evaluate their properties and effectiveness. For example, DNA nanostructures based on the branched and tetrahedron scaffolds were incubated in cell culture medium and both forms were found to be stable for at least an hour (FIG. 6). The DNA-tetrahedron was prepared by methods described in Zhang, et al., Chem Commun, 46, 6792-6794 (2010). Additionally, cellular uptake in a mouse macrophage-like cell line (RAW cells) demonstrated the DNA nanostructures enhanced uptake as compared to free antigen (FIG. 7). The immunogenesity of the CpG-tetrahedron-streptavidin complex may also be compared to CpG-J1-streptavidin complex in vivo (“J1” is also called a “DNA-branch”). Additionally, the long term memory responses in the mice injected with different CpG-DNA nanostructures may be monitored.

Thus, DNA-nanostructures can function as a synthetic platform for vaccine construction and provide a new line of vaccines against many different diseases.

### Example 2

### Novel HIV-Vaccines Built on DNA-Nanoparticles

The feasibility of using DNA-nanotechnology to rationally design and create more effective prophylactic HW vaccine candidates is described herein. Additionally, this novel approach may be extended, for example, to the vaccine development against other infectious agents, tumors and even addictive substances.

The modest success of the recent Thai RV-144 clinical trial, which only offered 31% protection from HW transmission among high risk groups, highlights an urgent need for new strategies in designing HW vaccines. Given the general consensus on the generation of neutralizing antibodies as an important correlate for protective immunity against HIV, some recent effort has been directed toward identifying neutralizing epitopes and displaying these epitopes onto a protein scaffold. This approach led to the production of antibodies resembling some aspects of neutralizing antibodies, but has still failed to neutralize HIV, indicating more work is needed to design and engineer “ideal neutralizing epitopes.”

Multivalent and multi-functional DNA-nanovaccines that enable targeting and engagement of B cells with other immune cells for an effective induction of a protective anti-HIV antibody immunity are described herein. Through multidisciplinary interactions and collaborations among virologists, immunologists, protein chemists, DNA-nanostructural chemists and bioinformatics scientists, a new strategy to design and construct HW vaccines may be developed. Specifically, by taking advantage of the programmable and addressable features of DNA-nanostructures, important biomolecules, including B cell epitopes of HIV glycoprotein, gp120/gp41, glycans, T helper-peptides, and adjuvant molecules may be assembled onto DNA-nanostructures at designated positions, i.e., in desired multi-valence, appropriate stoichiometry, and spatial orientations to elicit strong memory B cell responses against key epitopes of gp120/gp41. By combining systems biology approaches in vaccine design, including computational analyses of gp120/41 epitope sequences, protein 3-dimensional modeling, and glycan and peptide engrafting, with a novel DNA-nanoscaffold that empowers a controllable assembly of various epitopes and adjuvant molecules, immunogenic HIV-DNA origami that induce effective anti-HW antibody responses can be designed, constructed, selected and identified. This vaccine platform may induce a long-term production of multiple clones of high affinity neutralizing and/or inhibitory anti-HIV antibodies. Furthermore, the feasibility of the DNA-origami platform in constructing both prophylactic vaccines and anti-HIV DNA-scaffolds can lead to new lines of therapeutics for combating both active and latent HIV infections.

Self-assembling DNA-nanostructures may be used to engineer two known neutralizing epitopes. One is a peptide mimicking CD4 binding site (CD4b) that is recognized by the monoclonal antibody (mAb), b12, while the other is a glycan that binds to another neutralizing mAb, 2G12. These epitopes are grafted at defined positions, distances and configurations to mimic trimetric CD4bs or desired glycan structures. In addition, T helper-peptides and CpG oligonucleotides are assembled onto the surface of the proposed DNA-nanostructure at designated positions apart from the B or T cell epitopes. The proposed multi-valent and multi-functional DNA-nanoparticles resemble viral like particles (VLPs) by recruiting gp120-specific B cells, T helper cells and dendritic cells to the same microenvironment for their interactions and subsequent activation, which helps elicit a strong T-cell dependent B cell responses. However, as compared to the VLP assembled through viral capsid proteins, the present DNA-nanoparticles offer additional advantages: 1) more versatile and robust to assemble different antigenic components without complicated genetic engineering; 2) more precise control over the placement of various antigenic and adjuvant components onto the DNA-scaffolds; 3) relatively inert nature of DNA-scaffolds and lack of unrelated immunogenic viral proteins, which presents less likelihood to elicit non-target immune reactions that may cause deviation from the desired anti-HIV immune responses; and 4) potential function as polyreactive components to synergize the targeting of B cells with natural polyreactivity through heteroligation scheme, as described by Mouquet et al., Nature 467:591-596 (2010).

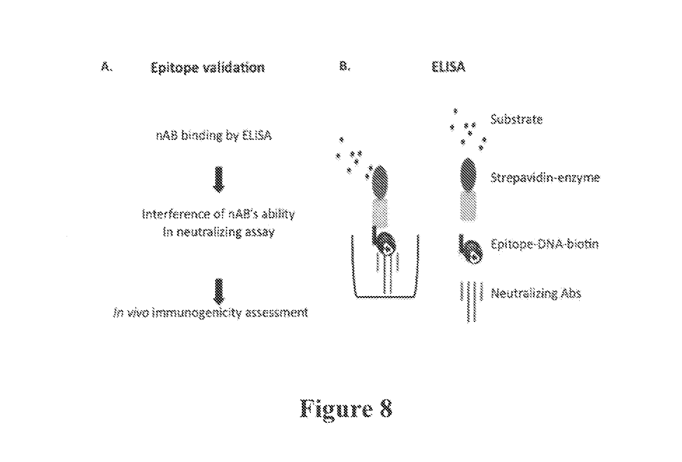

As a first step in the production of an HIV-nanovaccine, the feasibility of DNA-nanoscaffolds to elicit anti-HIV neutralizing antibody responses is investigated. Thus, previously reported neutralizing epitopes are used for the vaccine construction. It is determined whether the epitopes assembled onto the DNA-nanostructure retain the same configurations to elicit B cell responses with the generation of neutralizing antibody responses. Specifically, based on the reported crystal structure and 3D modeling of gp120 neutralizing epitopes, artificial model peptides are designed and engrafted in trimeric form onto DNA-nanoscaffolds. The conformation, structural integrity and immunogenicity of these neutralizing epitopes presented on the DNA-nanoparticles are tested by several assays, as outlined in FIG. 8A: 1) direct demonstration of their interaction with neutralizing antibodies; 2) interference with the ability of neutralizing antibodies in a neutralizing assay; and 3) assessment of anti-gp120 antibody response induced by the epitope-DNA-nanoparticles that also contain T-helper epitope and adjuvants, as illustrated in FIG. 2. In particular, a modified enzyme linked immunoabsorbent assay (ELISA) is employed for such analysis, in which known neutralizing antibodies, b12 or 2G12, coated to the plate are incubated with assembled epitope-DNA nanoparticles that contain biotin, followed by the strepavidin-enzyme mediated detection, as illustrated in FIG. 8B. Using this assay, it can be assessed whether the epitopes linked to the DNA-nanoparticles are functional. If indeed, these epitopes display the expected interactions with their specific neutralizing antibody, they are further tested for their action as an inhibitor in a well-established TZM-BI based neutralizing assay, in which a pre-incubation of neutralizing antibodies with the HIV-DNA-nanoparticles is conducted before adding to the mixture of pseudo-virus particles and a luciferase-based reporter line. Once the structures of the epitopes assembled onto the DNA-nanoscaffolds are validated as neutralizing epitopes, the in vivo immunogenicity of these epitopes is determined. Specifically, Balb/c mice are immunized with the constructed HIV-DNA particles that contain CD4b epitopes, T-helper epitopes and adjuvants, e.g., CpG oligonculeotides, which aims to induce local interactions among B, DC and Th cells to elicit an effective T-cell dependent humoral immune response. The serum from immunized mice is tested for the generation of neutralizing antibodies using TZM-BI neutralizing assay. The scope of epitopes in the DNA-based vaccine is expanded to also include additional sets of neutralizing epitopes to enhance the breadth and spectrum of B cell responses.

Subsequently, to further test the possible protective activity of the vaccine candidates, a humanized mouse model, wherein severe combined immunodeficient mice with mutations in both RAG2 and IL2-gamma-chain, i.e., RAG2-/-γc-/-, are reconstituted with human immune cells, is used. This humanized mouse model has been reported to recapitulate some aspects of HW immuno-pathogenesis and testing HW transmission via vaginal and rectal routes has been feasible. Thus, this model to evaluate the efficacy of the DNA-assembled HIV vaccines in reducing HIV infection is used before moving into the non-human primate system.

### Example 3

### Assembly of Systems-Biology Selected Epitopes onto Controllable DNA-Origami

Substance abuse is known to contribute to the transmission of human immunodeficiency virus type 1 (HIV-1) among adolescents and young adults. While a high HIV prevalence among IV-drug users is caused by direct exposure to HIV-contaminated blood through needle sharing, many drug users, including those using non-injecting substances, may also acquire HIV through risky sexual behaviors influenced by illicit drugs. Despite some success of several HIV prevention programs, such as clean needle exchange and safe-sex education, and powerful anti-retroviral drugs in reducing HIV transmission, an HIV vaccine may ultimately be our best hope for eradicating HIV/AIDS in high-risk drug user populations. Given the extremely high mutation rate of HIV genomes, only the prophylactic HIV vaccines that can induce immunity at the portal of entry would be considered valuable in controlling HW infection. Despite three decades of extensive effort, such vaccines are still not yet within our reach.

The modest success of one recent human HIV vaccine clinical trial (RV-144), which reduced the risk of HIV infection by 31 percent among a high-risk group in Thailand, raises hopes for making effective prophylactic HIV vaccines. Although the reason for such low efficacy and the mechanism underlying this modest protection remain elusive, anti-envelope antibodies and T-cell responses against HIV have been implicated in offering protective immunity (McElrath, et al., 2010, Immunity 33:542-554; Lu, et al., 2010, Curr HIV Res 8:622-629). A recent molecular characterization of gp120/gp41 proteins and their interactions with various antibodies leads to a general consensus that the production of a broad spectrum of neutralizing and/or inhibitory anti-gp120/gp41 antibodies is an important immunological correlate in preventing the establishment of HIV infection. However, major challenges in translating this fundamental knowledge into creating an effective HW vaccine still exist, including (1) how to identify neutralizing/inhibitory epitopes that are highly conserved across various HIV clades and strains and (2) how to construct these epitopes in such a way that they are immunogenic, poly-responsive, and primed for long-term production of high affinity anti-HW antibodies.

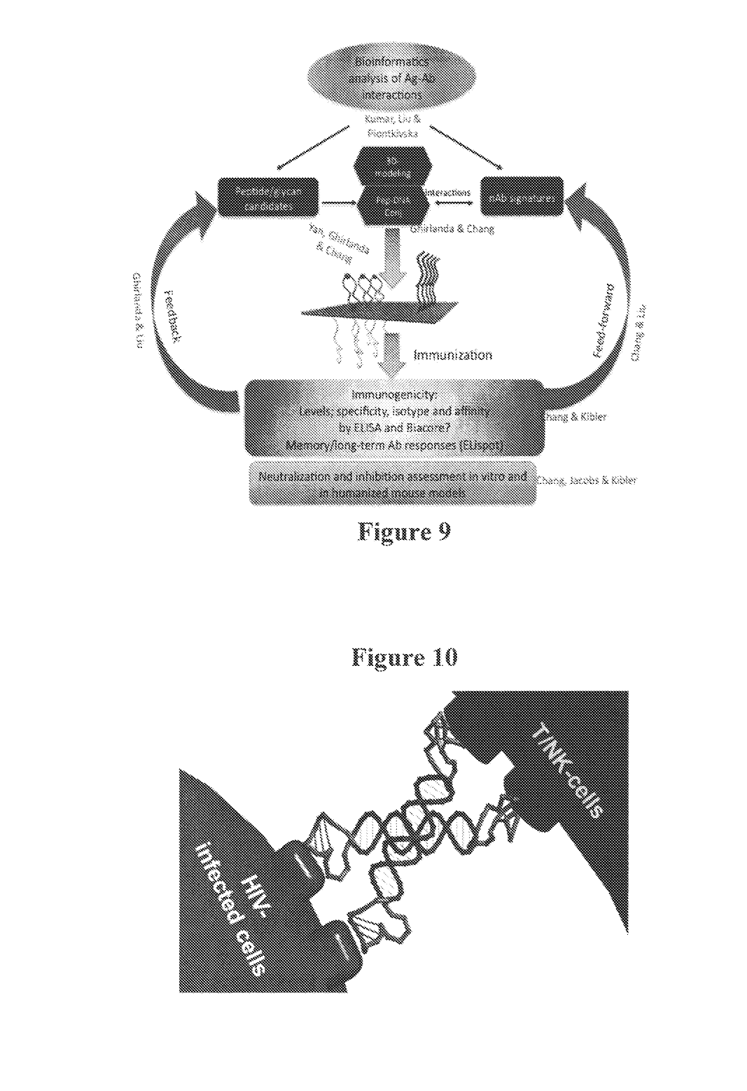

To meet these challenges, a new strategy to design and construct HIV vaccines is described herein. DNA nanotechnology has recently demonstrated its power in organizing various biomolecules. As an elegant bottoms-up method, DNA self-assembly based on a simple Watson-Crick principle has the inherent advantage of generating programmable nanostructures with nanometer precision in addressability (Seeman, N. C. 2003, Chem Biol 10:1151-1159). Assembly of various biomolecules onto a DNA-nanostructure can be systematically investigated with precise control over valences, configurations, and spatial distances. In addition to creating a DNA-origami detection array (Ke, et al., 2008, Science 319:180-183), a proof-of-concept DNA-nanoscaffold antigen was constructed and its feasibility to generate a strong memory IgG response against a model antigen, streptavidin (STV), assembled onto a DNA-nanoscaffold that contains CpG-oligonucleotides as adjuvants, was demonstrated (FIG. 3; see also Example 1). Tunable DNA structures to construct an HIV antibody vaccine is also explored, as described herein. By combining systems biology approaches in vaccine design, including computational analyses of gp120/gp41 epitope sequences, protein 3-dimensional modeling and peptide/glycan engrafting, with our novel DNA-nanoscaffold that empowers a controllable assembly of various epitopes and adjuvant molecules, immunogenic HIV-DNA origami that induces effective antibody for anti-HIV responses can be rationally designed, constructed, selected, and identified (FIG. 9). In addition, the size of antigen-assembled DNA-nanostructures could be controlled at 100 nm, an optimal size for antigen delivery and targeting to lymphoid tissues, especially to B cells (Bachmann, et al., 2010, Nat Rev Immunol 10:787-796; Elgueta, et al., 2010, Immunol Rev 236:139-150), which resembles a virus-like particle (VLP), but with much more robust capability than a VLP in antigen construction. The B-cell directed targeting helps the generation of long-term memory B cells (Bachmann, et al., 2010, Nat Rev Immunol 10:787-796; Elgueta, et al., 2010, Immunol Rev 236:139-150). Furthermore, unlike VLP, DNA-scaffolds are relatively inert, inducing minimal immune responses (Roberts, et al., 2011, Immunol Cell Biol. 89(4):517-25), and therefore, causing little interference with the desired anti-HIV immunity. Thus, the vaccine platform is superior over the conventional vaccines, as it allows rationale design of epitopes, robustness in antigen assembly, optimal particle size for antigen delivery, and weak immunogenicity of scaffolding DNA for causing low harm or interferences.

The experiments described herein may provide a window of opportunity for generating new sets of anti-HIV antibodies, and therefore, increasing the breadth and spectrum of anti-HIV antibodies. For example, the antibody elicited by antigen assembled onto DNA-nanoscaffolds may be directed toward epitopes that may not usually be displayed by conventional vaccines, which are based on protein-components or expressed through plasmids or viral vectors. Thus, by combining expertise from DNA and glycoprotein structural chemistry, bioinformatics and computational analyses with HIV virology and B cell biology, we aim to improve the efficacy of anti-HIV antibody responses to reduce acquisition and/or establishment of the infection by HIV or their mutant variants.

The antibody response elicited by these designed vaccines is first tested in a mouse model. In addition to measuring antibody levels, subclasses, and specificity, whether the generated antibodies display neutralizing activity using several cellular HIV models is also determined. Subsequently, to further test the possible protective activity of the vaccine candidates, a humanized mouse model is used, wherein severe combined immunodificient mice with mutations in both RAG2 and INF-gamma-chain, i.e., RAG2-/-γc-/-, are reconstituted with human immune cells. This humanized mouse model has been reported to recapitulate some aspects of HW immuno-pathogenesis feasible for testing HIV transmission via vaginal and rectal routes (Van Duyne, et al., 2009, Retrovirology 6:76). Therefore, this model is used to evaluate the efficacy of the DNA-assembled HIV vaccines in reducing HIV infection.

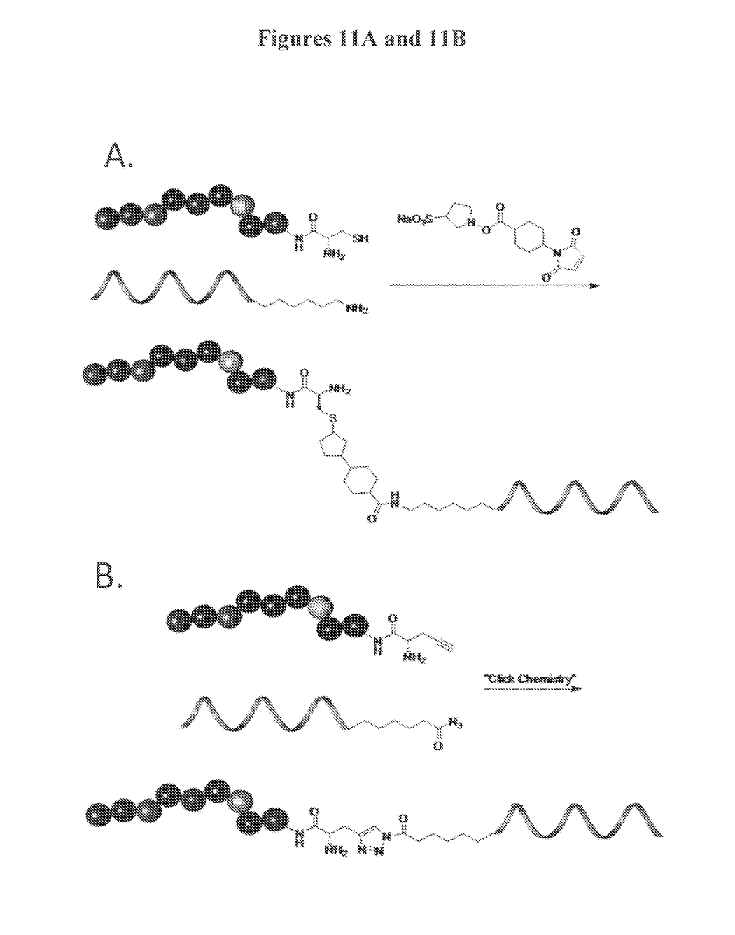

These studies aim to generate anti-HIV antibody vaccines that produce long-term memory antibody responses with broad-spectrum antibody specificities, provide a long lasting protection against HIV. This strategy may also be extended to develop T-cell vaccines against HIV, given the feasibility and robust nature of DNA-origami in epitope grafting, including T-cell epitopes and T-cell activation motifs. Furthermore, by engineering various targeting molecules onto DNA-nanoscaffolds in polymeric forms, e.g., HW-binding epitope (like gp120 binding aptamers), T-cell binding, or NK-cell binding molecules, various therapeutics can also be built to modulate the immune system and target HIV-infected cells for specific destruction. In addition to treating active infection, this strategy can also be used to tackle latent HIV infections by combining the proposed bi-specific DNA-targeting scaffolds (FIG. 10) with activation agents for T cells or macrophages that serve as an HIV reservoir. For this purpose, the EGFP-tagged chronically infected HIV cell line, THP89GFP cell line (kindly provided by Dr. Levy at NYU), is used as a testing model, since this line has been shown to expresses GFP upon HIV reactivation, which allows real-time monitoring of the reactivation process (Kutsch, et al., 2002, J Virol 76:8776-8786). By including the NK92 cell line along with bi-specific DNA-targeting, it can be determined whether GFP+-HIV reactivated cells are targeted for cell death. The in vivo efficacy assessment of these therapeutic DNA-nanoscaffolds could be conducted in the humanized RAG2-/-γc-/-model.

Innovative Platform for Developing HIV Vaccines and anti-HIV Therapeutics. Unlike conventional subunit vaccines and vector-based vaccines, which have been made empirically and often rely on slim chances that the protein or cells present immunogenic epitopes, this new vaccine platform empowers the rational design and robust synthesis of HIV antibody vaccines. Specifically, computational analyses of DNA/glycoprotein structures are applied for epitope identification, novel addressable DNA-nanoscaffolds are explored for epitope construction. A high-throughput antibody profiling and signature screening is employed for the selection of HIV antibody vaccines with “well-fit” epitopes to increase the spectrum and avidity of anti-HIV antibody responses. The capability to profile antigen-specific memory B cells allows for the identification of parameters and required components for generating long-term antibody responses. Furthermore, the feasibility of the DNA-origami platform in constructing both prophylactic vaccines and anti-HIV DNA-scaffolds (as shown in FIG. 10) leads to new lines of therapeutics for combating both active and latent HIV infections.

### Example 4

### A DNA Nanostructure Platform for Directed Assembly of Synthetic Vaccines

The goal of developing safer and more effective vaccines has been a priority since human beings began fighting disease through vaccination over 1000 years ago. Many of the vaccines that are currently administered were derived from live attenuated organisms, killed whole organisms, or subunit vaccines. Although live vaccines have the advantage of inducing a strong immune response, there is a risk that the attenuated organism will revert back to a virulent form, which is detrimental to the public health. Killed or inactivated whole organisms and subunit vaccines do not pose the same serious health risk; however, they tend to induce weaker or ineffective immune responses and often require multiple doses for enhanced efficacy. Recombinant DNA technology has facilitated the assembly of subunit proteins into virus like particles (VLPs) that resemble the structure of natural viruses but without containing their genetic material, representing a major breakthrough in vaccine development. Immunogenic epitopes displayed from the VLPs were shown to induce a strong immune response and thus, VLPs have been extensively explored as an effective and safe platform to assemble the epitopes of interest against many pathogens and tumor cells. However, sometimes, it is challenging to incorporate antigenic epitopes into VLPs at defined positions and configurations because of the inherent uncertainties in engineering epitope-VLP fusion proteins.

Alternatively, nanotechnology provides researchers with a robust platform for the assembly of subunit vaccines. In particular, biodegradable polymers such as poly(D,L-lactide-co-glycolide) (PLGA) have been used to encapsulate vaccine antigens and adjuvants. These subunit vaccines have been shown to increase antigen delivery and antigen presenting cell (APC) targeting, thereby enhancing the immunogenicity of the antigen. Today, DNA nanotechnology is recognized as a highly programmable and robust way to self-assemble heterogeneous nanostructures. A variety of different two- and three-dimensional DNA nanostructures (Seeman, N. C. J Theor Biol 1982, 99, 237-247; Rothemund, P. W. Nature 2006, 440, 297-302; Shih, W. M.; Quispe, J. D.; Joyce, G. F. Nature 2004, 427, 618-621; Zhang, C.; Su, M.; He, Y.; Zhao, X.; Fang, P. A.; Ribbe, A. E.; Jiang, W.; Mao, C. Proc Natl Acad Sci USA 2008, 105, 10665-10669; Douglas, S. M.; Dietz, H.; Liedl, T.; Högberg, B.; Graf, F.; Shih, W. M. Nature 2010, 459, 414-418; Han, D.; Pal, S.; Nangreave, J.; Deng, Z.; Liu, Y.; Yan, H. Science 2011, 332, 342-346; Dietz, H.; Douglas, S. M.; Shih, W. M. Science 2009, 325, 725-730) have been constructed and used for precisely organizing biochemical molecules (Auyeung, E.; Cutler, J. I.; Macfarlane, R. J.; Jones, M. R.; Wu, J. S.; Liu, G.; Zhang, K.; Osberg, K. D.; Mirkin, C. A. Nature Nanotechnology 2012, 7, 24-28; Aldaye, F. A.; Palmer, A. L.; Sleiman, H. F. Science 2008, 321, 1795-1799; Chhabra, R.; Sharma, J.; Liu, Y.; Rinker, S.; Yan, H. Adv Drug Deliv Rev 2010, 62, 617-625; Yan, H.; Park, S. H.; Finkelstein, G.; Reif, J. H.; LaBean, T. H. Science 2003, 301, 1882-1884) and targeted cellular transport and delivery (Walsh, A. S.; Yin, H.; Erben, C. M.; Wood, M. J.; Turberfield, A. J. ACS Nano 2011, 5, 5427-5432; Douglas, S. M.; Bachelet, I.; Church, G. M. Science 2012, 335, 831-834; Surana, S.; Bhat, J. M.; Koushika, S. P.; Krishnan, Y. Nat Commun 2011, 2, 340). Gaining control over structural features such as particle size and shape, epitope valency, and configuration is highly desirable and long sought after in vaccine development and DNA nanostructures present an opportunity to exert such control. Several research groups have assembled multiple adjuvant elements on a DNA nanostructure and found increased immunostimulation in vitro and ex vivo (Li, J.; Pei, H.; Zhu, B.; Liang, L.; Wei, M.; He, Y.; Chen, N.; Li, D.; Huang, Q.; Fan, C. ACS Nano 2011, 5, 8783-8789; Schuller, V. J.; Heidegger, S.; Sandholzer, N.; Nickels, P. C.; Suhartha, N. A.; Endres, S.; Bourquin, C.; Liedl, T. ACS Nano 2011, 5, 9696-9702). Here we provide the first evidence that antigens and adjuvants assembled by DNA nanostructures induce strong antibody responses in vivo, highlighting the potential of DNA-nanostructures to serve as new platforms for vaccine construction.

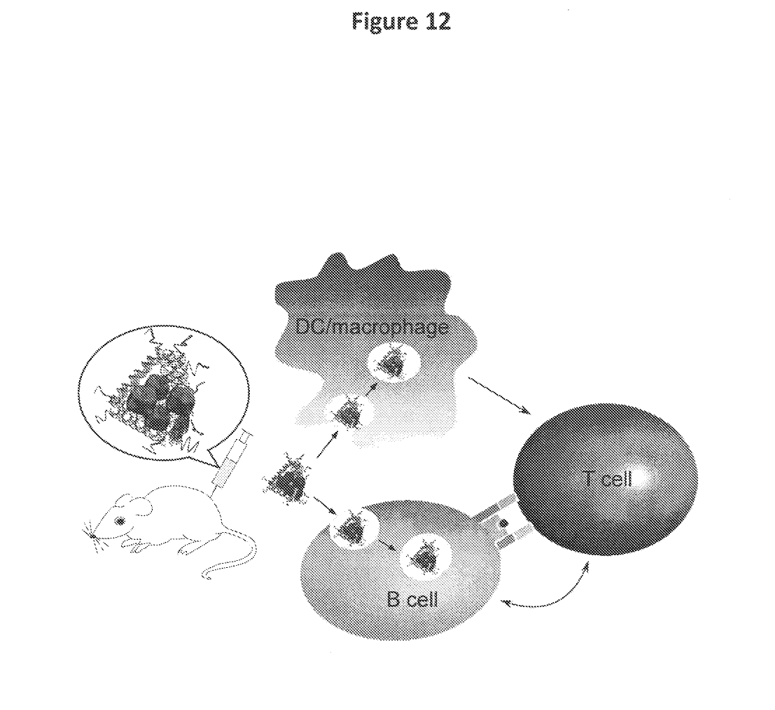

We used a tetrahedral DNA nanostructure (Zhang, C.; Su, M.; He, Y.; Leng, Y.; Ribbe, A. E.; Wang, G.; Jiang, W.; Mao, C. Chem Commun (Camb) 2010, 46, 6792-6794; Zhang, C.; Tian, C.; Guo, F.; Liu, Z.; Jiang, W.; Mao, C. Angew Chem Int Ed Engl 2012) as a scaffold to assemble a model antigen, streptavidin (STV), and a representative adjuvant, CpG ODN (Klinman, D. M. Nat Rev Immunol 2004, 4, 249-258), into a synthetic vaccine complex (FIG. 12). This vaccine complex resembles a natural viral particle in both size and shape (Zhang, C.; Tian, C.; Guo, F.; Liu, Z.; Jiang, W.; Mao, C. Angew Chem Int Ed Engl 2012; Bachmann, M. F.; Jennings, G. T. Nat Rev Immunol 2010, 10, 787-796), where the STV and CpG ODN elements are located at particular positions (FIG. 12 and Figure S1). The complex was tested both in vitro and in vivo for its immunogenicity, particularly its ability to elicit an antibody response against the model antigen, STV.

Targeted delivery of the antigen to antigen presenting cells, including macrophages, dendritic cells (DCs) and B cells, is a vital first step in initiating an effective immune response. Previous studies have shown that the size, shape, surface charge, hydrophobicity, hydrophilicity, and receptor interactions of an antigen can influence its uptake by APCs (Bachmann, M. F.; Jennings, G. T. Nat Rev Immunol 2010, 10, 787-796). After internalization, the targets are processed and presented to T cells for T cell activation. It has been demonstrated that co-localization of antigens and adjuvants within the same APCs can augment antigen presentation and T cell activation (Krishnamachari, Y.; Salem, A. K. Adv Drug Deliv Rev 2009, 61, 205-217). Finally, activated T cells assist in the differentiation of antigen-specific B cells and the production of the antibodies that are specific to the target antigen, as illustrated in FIG. 12. Given the recent report that DNA nanostructures increase the amount of CpG adjuvant molecules that are internalized by APCs (Li, J.; Pei, H.; Zhu, B.; Liang, L.; Wei, M.; He, Y.; Chen, N.; Li, D.; Huang, Q.; Fan, C. ACS Nano 2011, 5, 8783-8789; Schuller, V. J.; Heidegger, S.; Sandholzer, N.; Nickels, P. C.; Suhartha, N. A.; Endres, S.; Bourquin, C.; Liedl, T. ACS Nano 2011, 5, 9696-9702), we speculated that DNA nanostructures would also increase the amount of antigen taken by APCs, thereby promoting co-delivery of the antigen and CpG to the same APC population.

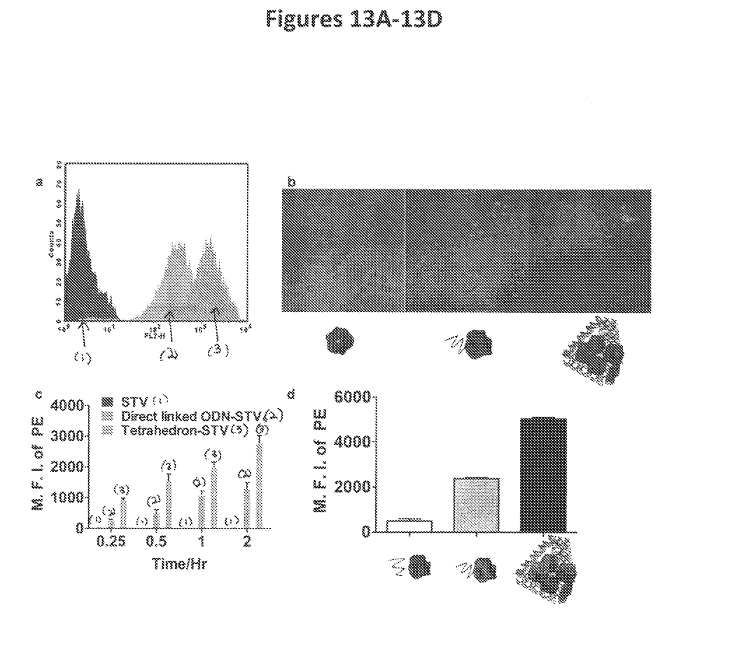

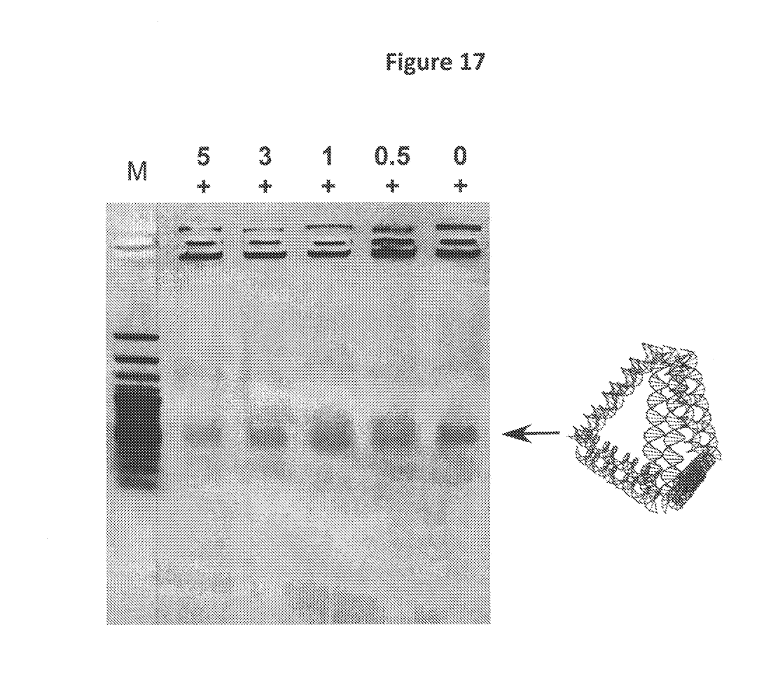

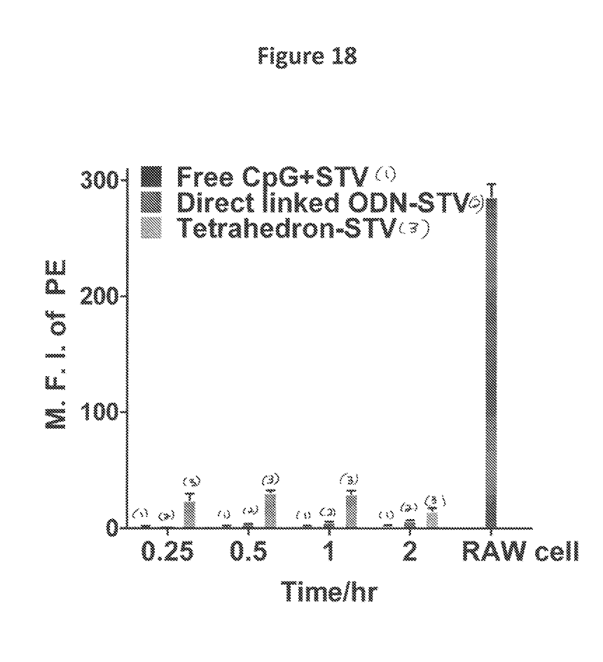

To test this hypothesis we loaded fluorescently labeled model antigen, phycoerythrin conjugated streptavidin (PE-STV), onto the DNA tetrahedron and used flow cytometry to track the internalization of the complex in a mouse macrophage-like cell line (RAW 264.7). As shown in FIGS. 13a and 13c, internalization of the tetrahedron-PE-STV complex occurs quickly (within 15 minutes) in the RAW 264.7 cells. Confocal microscope analysis (Supplementary Information “SI”) of the sample confirmed that the PE fluorescent signal was present inside the cells (FIG. 13b). The fluorescent signal in the tetrahedron-PE-STV group continued to increase up to 6 hours, while no fluorescent increase was observed in the control group treated with only PE-STV (FIG. 13c). This result indicates that the DNA scaffold enhances cellular uptake of the antigen. This finding was further substantiated in primary DCs (FIG. 13d, details in SI), but not in a mouse B cell line that lacked the specific antibody required to bind STV (FIG. 18). Furthermore, the tetrahedron scaffolded antigen complex was shown to be quite stable in the presence of serum (FIG. 17), which may be sufficient for in vivo capture by APCs. Our in vitro study, together with previous reports of DNA tetrahedron facilitating adjuvant uptake, suggest that DNA nanostructures can promote delivery of both assembled antigens and adjuvants to APCs, which is a prerequisite for induction of an effective immune response.

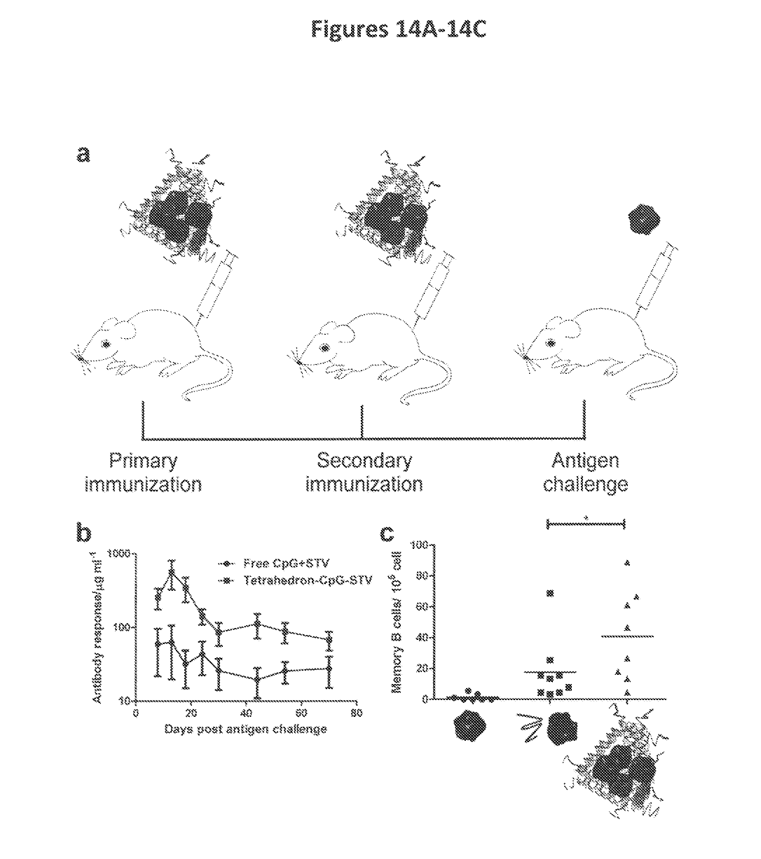

We next compared the immunogenicity of the fully assembled tetrahedron-STV-CpG ODN vaccine complexes in inducing anti-STV antibody responses in a BALB/c mouse model to those of an unassembled mixture of STV and CpG ODN, or STV alone. Specifically, we followed the antibody response in three groups injected with different combinations of CpG ODN and STV: 1) STV only; 2) free STV mixed with CpG; and 3)tetrahedron-STV-CpG ODN complex. As outlined in FIG. 14a, after two immunizations with the DNA scaffolded vaccine complex followed by a challenge of STV protein only, serum was collected from each mouse group and the level of anti-STV IgG antibodies was assessed using an enzyme-linked immunosorbent assay (ELISA). Over a period of 70 days, we found that mice immunized with the fully assembled tetrahedron-STV-CpG ODN complex developed a much higher level of anti-STV IgGs than the free CpG+STV (FIG. 14b). This reflects the development of long-term immunity against the antigen, presumably due to the persistence of long-lived antibody secreting plasma cells and/or generation of STV-specific memory B cells.

To directly evaluate the long-term immunity induced by various immunization regimes, we applied an enzyme-linked immunosorbent spot (ELISPOT) assay that allows numeration of STV-specific memory B cells present in the spleen cells of immunized mice. Specifically, after in vitro stimulation with STV, memory B cells are converted into antibody-secreting cells (ASCs) which are detected by the ELISPOT assay. As shown in FIG. 14c, significantly elevated levels of specific ASCs were found in mice immunized with the tetrahedron-STV-CpG ODN complex compared to those immunized with free CpG+STV and STV only. Thus, the tetrahedron scaffolded-STV-CpG ODN complexes induce a stronger and longer lasting anti-STV antibody response, due in part to the generation of STY-specific memory B cells.

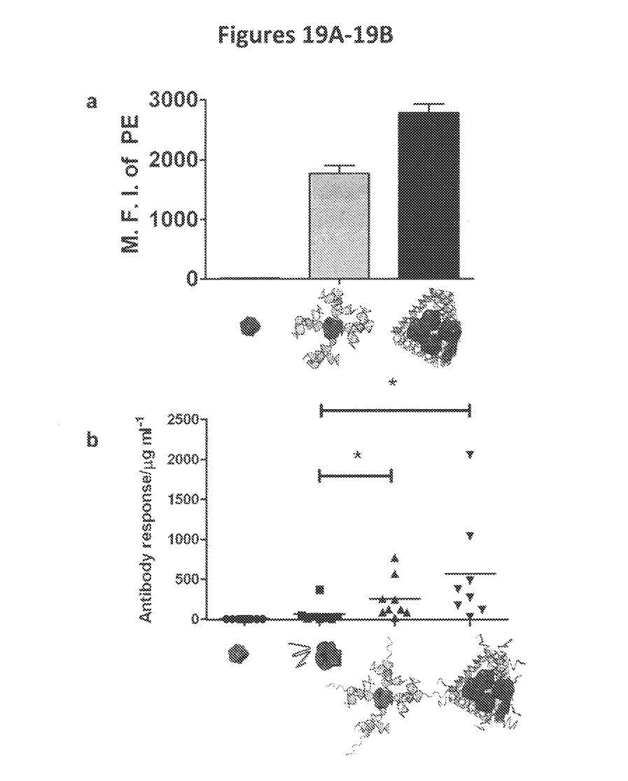

Beyond the tetrahedral DNA nanostructure described above, we also constructed a branch-shaped structure for antigen-adjuvant co-assembly (FIG. 19). The antigen assembled by this branched DNA structure induces an antibody response at a level intermediate between that of free CpG+STV and the tetrahedron-STV-CpG ODN complex (FIG. 19). Interestingly, in the in vitro experiment antigen internalization for the branch-STV complex is lower than for the tetrahedron-STV complex, but higher than for free STV. Taken together, the different DNA nanostructures appear to influence both the in vitro cellular uptake of the antigen, and the in vivo induction of antigen-specific antibody responses. This is likely because of differences in the size, shape or stability of the DNA nanostructures which may affect their ability to deliver the attached antigen and adjuvant to APCs. While the actual mechanisms still remain to be elucidated, the observed correlation between an elevated level of antigen internalization and a stronger antibody response may provide us with a screening tool to predict or identify the optimal DNA nanostructures for subsequent vaccine construction and test in vivo.

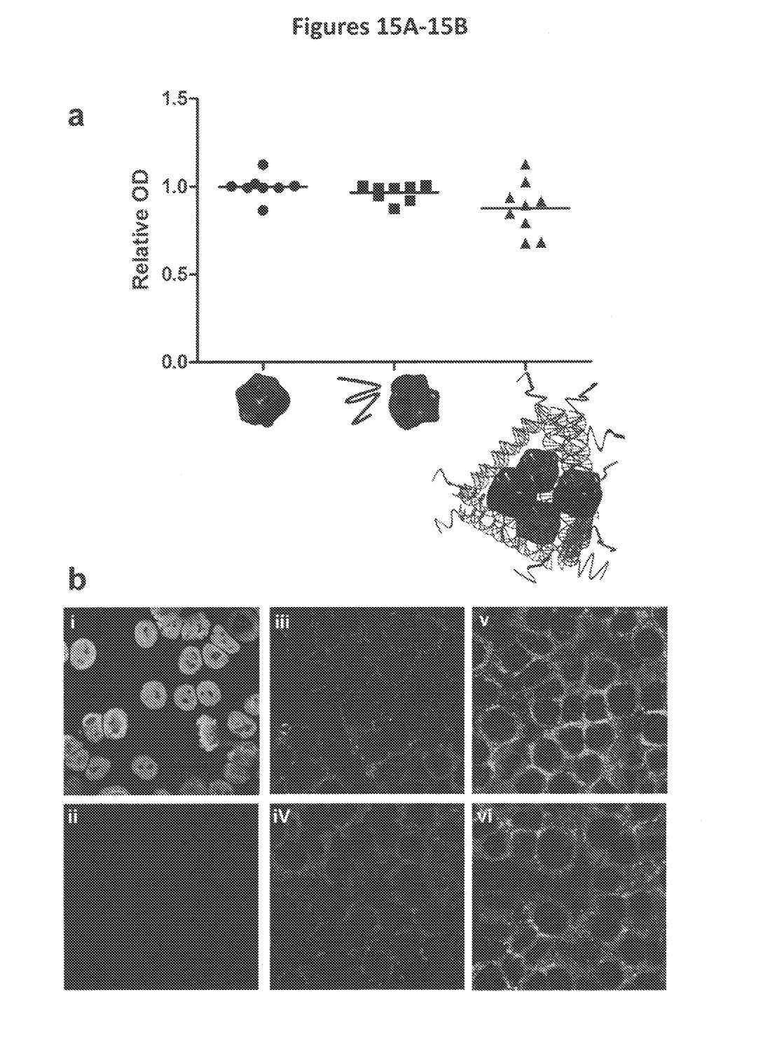

In addition to efficacy, the safety of a vaccine platform is another important parameter in vaccine design. Any non-targeted immune responses, including those against the platform itself, should be minimized. We should point out that the amount of antigen and CpG ODN used in our antigen-adjuvant-DNA complex to induce a specific immune response is lower than reported elsewhere, implying the reduced chance of this complex to cause overt non-specific activation often associated with injection of free adjuvant (Klinman, D. M.; Barnhart, K. M.; Conover, J. Vaccine 1999, 17, 19-25). Furthermore, any immune reaction mounted against the double stranded DNA scaffold could result in tissue damage and trigger autoimmunity; for example, anti-double stranded DNA (anti-dsDNA) antibodies are implicated in the pathogenesis of many autoimmune diseases including systemic lupus erythematosus. We measured the level of anti-dsDNA antibodies in the mouse serum 18 days post-secondary immunization, a time when the anti-STV antibody level was still very high and anti-dsDNA antibodies, if present, would be detected with the highest sensitivity. Using two independent methods, we observed no detectable level of anti-dsDNA antibodies in the tetrahedron-STV-CpG ODN group (FIG. 15).

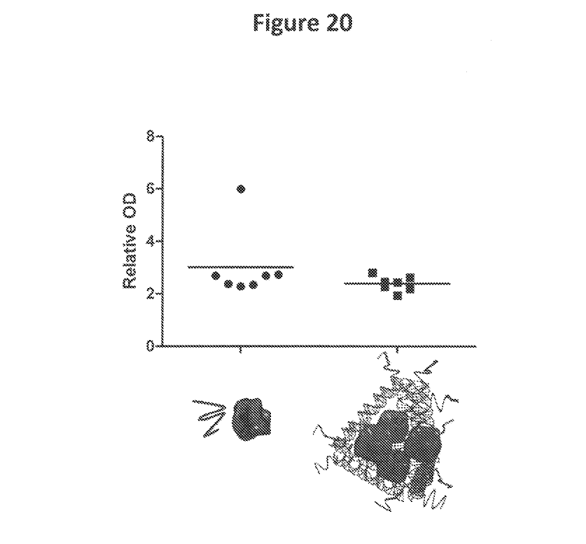

In addition to the test of anti-dsDNA antibody, we used ELISA analysis to investigate whether there is any antibody generated against the tetrahedron-shaped structure. Similarly, no antibody was detected in the mouse serum 18 days post-secondary immunization (FIG. 20). Taken together, these results indicate that the antigen-adjuvant-DNA complex is relatively safe and that the response induced by the vaccine complex is specific to the antigen and not the DNA platform.

In summary, we demonstrated that a DNA scaffold can be used to construct an antigen-adjuvant complex that elicits a strong and specific antibody response in vivo, without inducing an undesirable response against the scaffold itself. Programmable DNA nanostructures have several advantages over other vaccine platforms, including the ability to control the valency of the immunogenic elements and their spatial arrangement, which is critical to generation of effective humoral immune responses. With well-established protein-DNA conjugation techniques, it may be feasible to attach multiple antigen epitopes on a single DNA scaffold. The epitopes could be precisely arranged to facilitate optimal binding to specific B cell receptors. At the same time, there is the ability to assemble other immunogenic molecules or “danger signals” on the same DNA scaffold to enhance the immunogenicity of subunit vaccines without compromising the safety. This work demonstrates the potential of DNA nanostructures to serve as general platforms for vaccine development.

### Experimental Methods

Animals:

Female BALB/c mice were obtained from Charles River Laboratories and maintained in a pathogen-free animal facility at the Arizona State University Animal Resource Center. All mice were handled in accordance with the Animal Welfare Act and Arizona State University Institutional Animal Care and Use Committee (IACUC). Before experimental treatment, the mice were randomly distributed in cages and allowed to acclimate for at least 1 week before vaccination. Each 6-week old mouse was immunized subcutaneously with 10 μg streptavidin and/or 3.3 μg CpG ODN or equivalent amounts of CpG DNA incorporated into DNA scaffold on days 0 and day 27, and challenged intraperitoneally with 10 μg of streptavidin alone on day 51. Blood was subsequently collected from cheek veins in accordance with the Arizona State University IACUC.

Bone Marrow Derived Dendritic Cells (Primary DCs):

Mice were asphyxiated by CO₂ and bone marrow from the leg bone was extracted and depleted of red blood cells by an ACT lysis buffer (mix 90 mL of 0.16 M NH₄Cl and 10 mL of 0.17 M Tris (pH 7.65); the pH was then adjusted to 7.2 with 1 M HCl and sterilized. The washed bone marrow cells were cultured in Dulbecco's modified Eagles medium (DMEM, Sigma) supplemented with 10% heat-inactivated FBS, 1% penicillin-streptomycin, 1% glutamine, murine GM-CSF (10 ng/ml, Prospec), and murine IL-4 (10 ng/ml, Prospec) at 37° C. with 5% CO₂. After 4 days of growth, the medium was replaced with fresh DMEM supplemented with murine GM-CSF (10 ng/ml) and murine IL-4 (10 ng/ml). Cells were harvested on day 7 and seeded at a density of 2.5 10⁵ cells/well in U-bottom 96-well plates (CellStar) with DMEM supplemented with murine GM-CSF (20 ng/ml) and murine IL-4 (20 ng/ml) and held overnight before treatment.

Flow Cytometry:

Cells were collected from a culturing dish or well by pipetting or trypsinization, intensively washed with buffer (phosphate-buffered saline, 1% BSA, and 0.2% sodium azide), centrifuged at 380 g for 5 minutes, re-suspended in the buffer and analyzed by flow cytometry on a FACSCalibur (BD Biosciences). Data was analyzed using CellQuest (BD Biosciences).

Confocal Microscopy:

All fluorescent images were collected by a Plan-Neoflur 40/1.3 oil DIC at a working distance of 0.17 using a Zeiss LSM 510 laser scanning microscope (Carl Zeiss, Gottingen, Germany) connected to a LSM510 laser module with the following lasers: HeNe488 (both PE fluorescence and FITC fluorescence) and HeNe633 (transmitted light image). Fluorescence was recorded as square 8-bit images (1042 1042 pixels).

Antigen Internalization:

2 10⁵ RAW 264.7 cells (or A20 cells) were seeded on 24-well plates in 0.5 ml DMEM supplemented with 10% heat-inactivated FBS, 1% penicillin-streptomycin, 1% glutamine medium (or 0.5 ml RPMI-1640 medium, sigma, supplemented with 10% heat-inactivated FBS, 1% penicillin-streptomycin, 1% glutamine) and cultured overnight before treatment; the following day various combinations of free 2.5 μg PE-STV (BD Pharmingen) or equivalent, 0.825 μg CpG, and DNA scaffolded complexes were added to each well for various incubation times. The cells were subsequently trypsinized before analyzing by FACS Calibur (BD Biosciences, San Diego, Calif.). For primary DCs, 2.5 10⁵ cells were seeded on 96-well plates in 200 μl DMEM supplemented with murine GM-CSF (20 ng/ml), and murine IL-4 (20 ng/ml) overnight before treatment. The cells were subsequently treated with various combinations of 1 μg PE-STV or equivalent and 0.33 gig of CpG or equivalent for 2 hours. The cells were then either treated with trypsin or incubated with PE-AlexaFluor647 labeled anti-mouse CD11c antibodies; CD11c+ PE+ cells were analyzed by FACS Calibur (BD Biosciences, San Diego, Calif.).

Detection of Anti-STV IgG by ELISA:

Maxisorp® flat-bottom 96-well plates (Thermo) were coated with 1 μg/ml streptavidin (MP) in coating buffer (6.06 g/L Tris-base, 0.2 g/L NaN₃, pH 9.5) at room temperature and held overnight. The next day the plates were blocked by blocking buffer (10 g/L BSA, 0.1% NaN₃, 0.05% Tween-20 in PBS buffer) at 37° C. for 1 hour, followed by the addition of diluted mouse serum to each well and incubation for an additional 2 hours at 37° C. The presence of serum antibodies was then verified by adding alkaline phosphatase labeled goat anti-mouse IgG (Sigma) and subsequently 4-nitrophenyl phosphate disodium salt hexahydrate substrate (Sigma). The OD at 405 nm was measured using a microreader and the anti-STV IgG level was calculated by fitting the OD405 to a standard curve that was generated from a standard anti-STY antibody (GeneTex).

ELISPOT Assay:

Mice were asphyxiated by CO₂ and the spleen was extracted and depleted of red blood cells by an ACT lysis buffer (supplementary information). The washed spleen cells were incubated with 10 μg/ml streptavidin in RPMI-1640 media at 37° C. with 5% CO₂ for 72 hours, seeded on opaque MultiScreen^{HTS} 96-well Plates (Millipore) that were pre-coated with 5 μg/ml goat anti-mouse IgG (Invitrogen), and incubated for another 22 hours. The plates were thoroughly washed and the presence of spots was detected by adding alkaline phosphatase labeled streptavidin (Vector Laboratories) followed by the addition of BCIP/NBT substrate (Sigma).

Anti-dsDNA Antibody Detection:

A dsDNA ELISA kit (Calbiotech) and a microscope based anti-nuclear antibody kit (ANA kit, Antibodies Incorporated) was used to evaluate the level of anti-dsDNA antibodies present in mouse serum samples 18 days post injection. Detection was performed following manufactures' instruction with modifications to accommodate measurements in mice. Briefly, the secondary antibody in ELISA was replaced with an HRP-conjugated goat anti-mouse antibody, and the secondary antibody in the ANA kit was supplemented with alkaline phosphatase conjugated goat anti-mouse IgG. For the ANA kit, the mouse serum was diluted 20 times.

Anti-Tetrahedron-Shaped DNA Antibody Detection:

Maxisorp® flat-bottom 96-well plates (Thermo) were coated with 1 μg/ml avidin in coating buffer (6.06 g/L Tris-base, 0.2 g/L NaN₃, pH 9.5) at room temperature and held overnight. The next day the plates were blocked by blocking buffer (10 g/L BSA, 0.1% NaN₃, 0.05% Tween-20 in PBS buffer) at 37° C. for 1 hour, followed by the addition of 62.5 nM tetrahedron DNA in TAE/Mg²⁺ buffer and incubation at room temperature for 30 min. Then diluted mouse serum is added to each well and incubated for an additional 1 hours at room temperature. The presence of serum antibodies was then verified by adding HRP labeled goat anti-mouse antibody and TMB super sensitive one component HRP microwell substrate (BioFX). The OD at 650 nm was measured using a microreader and the relative OD was calculated by comparing to the OD650 of the negative control provided in the dsDNA ELISA kit (Calbiotech).

Statistical Analysis:

Prism 5.0 software (GraphPad) was used to analyze the antibody response, memory B cell response, and to determine the statistical significance of differences between groups (we applied a one-tailed unpaired student t test. P values <0.05 were considered significant).

DNA Strands:

All the DNA strands were purchased from Integrated DNA Technologies Inc. (CA), and the DNA strand sequences are listed as follows (* indicates phosphothioate bond):

Strand-L:
5′ AGG CAC CAT CGT AGG TTT C TTG CCA GGC ACC
ATC GTA GGT TTCT TGC CAG GCA CCA TCG TAG GTT
T CTT GCC 3′
Strand-M-linker:
5′ CAG AGG CGC TGC AAG CCT ACG ATG GAC ACG
GTA ACG ACT 3′
Strand-CpG linker:
5′ AGC AAC CTG CCT GTT AGC GCC TCT GTT TTT
T*C*C *A*T*G *A*C*G *T*T*C*C*T*G*A*C*G*T*T 3′
Strand-S:
5′/5Biosg/TTA CCG TGT GGT TGC TAG TCG TT 3′
CpG ODN:
5′ TCC ATG ACG TTC CTG ACG TT 3′
Biotin-CpG:
5′/5Biosg/T*C*C*A*T*G*A*C*G*T*T*C*C*T*G*A*C*
G*T*T 3′

All the component DNA strands were mixed at a ratio of 1:3:3:3 (L:M:CpG:S) in a Tris-acetic acid-EDTA-Mg2+ buffer and the mixture was slowly cooled from 95° C. to 4° C. over 48 hours. The assembled DNA structures were characterized by 3.5% non-denaturing PAGE at 20° C.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context. Thus, for example, reference to “a subject polypeptide” includes a plurality of such polypeptides and reference to “the agent” includes reference to one or more agents and equivalents thereof known to those skilled in the art, and so forth.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventor for carrying out the invention. Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

1. A composition comprising a DNA-nanostructure and at least one targeting moiety, wherein the at least one targeting moiety is linked to the DNA-nanostructure; and wherein the at least one targeting moiety is selected from the group consisting of antigens, aptamers, shRNAs and combinations thereof.

2. The composition of claim 1, wherein the DNA-nanostructure is selected from a biotin-oligo, a DNA-tetrahedron and a DNA-branch.

3. (canceled)

4. (canceled)

5. (canceled)

6. The composition of claim 2, wherein the DNA-nanostructure is a DNA-branch that comprises four oligonucleotides, wherein one oligonucleotide contains at least one CpG motif and/or wherein one oligonucleotide is biotinylated.

7. (canceled)

8. (canceled)

9. The composition of claim 1, wherein the composition further comprises at least one adjuvant, wherein the adjuvant is linked to the DNA nanostructure.

10. The composition of claim 9, wherein the at least one adjuvant is an oligonucleotide containing at least one immunostimulatory CpG motif.

11. (canceled)

12. The composition of claim 1, wherein the antigen is selected from the group consisting of B-cell epitopes, T-cell epitopes, Tₕₑₗₚₑᵣ epitopes, epitopes derived from gp120, gp41 epitopes, glycans, peptides, T-helper peptides, and streptavidin.

13. The composition of claim 12, wherein the antigen binds to a neutralizing antibody or an inhibitory antibody.

14. The composition of claim 1, wherein the targeting moiety is an antigen that is a neutralizing epitope, wherein the neutralizing epitope is a gp120 epitope, gp41 epitope, a CD4b epitope, a peptide that mimics the CD4 binding site (CD4b), a peptide that binds to the neutralizing antibody b12, or a glycan that binds to the neutralizing antibody 2G12.

15. (canceled)

16. (canceled)

17. (canceled)

18. (canceled)

19. The composition of claim 1, wherein the at least one targeting moiety is an aptamer.

20. The composition of claim 19, wherein the aptamer binds to an HIV epitope or a cell surface receptor expressed on an immune cell.

21. (canceled)

22. (canceled)

23. The composition of claim 1, wherein the at least one targeting moiety is shRNA.

24. The composition of claim 23, wherein the shRNA is a Foxop3-shRNA.

25. The composition of claim 1, wherein the composition comprises at least two targeting moieties.

26. (canceled)

27. (canceled)

28. The composition of claim 25, wherein one targeting moiety is a glycan that binds to the neutralizing antibody 2G12 and the other targeting moiety is a peptide that binds to the neutralizing antibody b12.

29. The composition of claim 28, further comprising at least one T-helper peptide and at least one adjuvant, wherein the T-helper peptide and the adjuvant are linked to the DNA nanostructure.

30. (canceled)

31. The composition of claim 25, wherein one targeting moiety is a first aptamer that binds to an HIV-infected cell and the other targeting moiety is a second aptamer that binds to binds to an immune cell.

32. (canceled)

33. The composition of claim 31, wherein the first aptamer binds to a gp120 epitope on the HIV-infected cell and the second aptamer binds to CD16 on the immune cell.

34. (canceled)

35. The composition of claim 25, wherein one targeting moiety is an aptamer and the other targeting moiety is shRNA.

36. The composition of claim 1, in combination with a physiologically-acceptable, non-toxic vehicle.

37. A method of inducing an immune response in a subject, comprising administering to the subject a therapeutically effective amount of the composition of claim 36.

38. A method of inducing the production of high affinity neutralizing antibodies or inhibitory antibodies comprising administering the composition of claim 36 to a subject having a pathological condition.

39. (canceled)

40. A method for treating a subject with a pathological condition comprising administering a therapeutically effective amount of the composition as described in claim 36 to the subject.

41. (canceled)

42. (canceled)

43. (canceled)

44. (canceled)

45. The method of claim 40, wherein the pathological condition is human immunodeficiency virus (HIV).

46. (canceled)
